(19) United States
(12) Patent Application Publication
(10) Pub. No.: US 2025/0325449 A1
(43) Pub. Date: Oct. 23, 2025

(54) DISPLACEMENT FILLING DEVICE FOR FILLING AN INTERIOR CAVITY OF A DRUG DELIVERY ASSEMBLY

(71) Applicant: Mott Corporation, Farmington, CT (US)

(72) Inventors: Alex Hill, Manchester, CT (US); David Gonthier, Middletown, CT (US)

(21) Appl. No.: 19/185,606

(22) Filed: Apr. 22, 2025

Related U.S. Application Data
(60) Provisional application No. 63/637,322, filed on Apr. 22, 2024.

(51) Int. Cl. A61J 1/20

(52) U.S. Cl. CPC A61J 1/201

(57) ABSTRACT

A filling system that supports a syringe with a drug and an implantable drug delivery system (IDDS), the filling system having: a filling fixture having a wall extending from a top to a bottom along a first axis and defining an outer wall surface and inner wall surface that are radially spaced from each other, wherein the inner wall surface defines a passage that includes: a top bore portion that is configured to seat the barrel bottom of the syringe; and a bottom bore portion configured to seat the ID DS such that the septum of the IDDS faces the syringe, wherein the top bore portion and the bottom bore portion are spaced apart from each other such that the needle of the syringe extends into the IDDS.

### CROSS REFERENCE

This application claims priority to US 63/637,322 filed on Apr. 22, 2024, the entire contents of which is incorporated herein by reference.

### FIELD OF THE DISCLOSURE

The present disclosure relates to needle displacers for filling a drug delivery system, in particular an implantable drug delivery system (or IDDS) for extended drug delivery and/or tunability and systems/methods for utilizing and fabricating the drug delivery system and, more particularly, to single or dual compartment, and dual porous membrane based (e.g., porous metal membrane based) drug delivery system for extended drug delivery (e.g., via passive diffusion) and/or tunability.

### BACKGROUND OF THE DISCLOSURE

In general, some needle displacers for filling drug delivery systems and drug delivery systems or the like are known.

An interest exists for improved filling devises for filling drug delivery systems, drug delivery, and related methods of use.

Opportunities for improvement are addressed and/or overcome by the assemblies, methods, and devices of the present disclosure.

### BRIEF SUMMARY OF THE DISCLOSURE

The present disclosure provides advantageous drug filling systems for filling drug delivery systems, drug delivery systems for extended drug delivery and/or tunability, and improved systems/methods for utilizing and fabricating the devices and the drug delivery systems. M ore particularly, the present disclosure provides single or dual compartment, and dual porous membrane based (e.g., porous metal membrane based) drug delivery systems for extended drug delivery (e.g., via passive diffusion) and/or tunability.

More specifically, disclosed is a first embodiment of a filling system that supports a syringe with a drug and an implantable drug delivery system (IDDS), wherein the syringe has a barrel extending from a barrel top to a barrel bottom, a plunger extending from the barrel top and a needle extending from the barrel bottom, and the IDDS defines a compartment with first and second ends that are opposite each other and a shell extending between the first and second ends, wherein: one of the first and second ends has a septum that is configured to receive the drug from the needle of the syringe; and one or more of another of the end first and second ends, and the shell, includes a porous surface, the filling system including: a filling fixture having a wall extending from a top to a bottom along a first axis and defining an outer wall surface and inner wall surface that are radially spaced from each other, wherein the inner wall surface defines a passage that includes: a top bore portion that is configured to seat the barrel bottom of the syringe; and a bottom bore portion configured to seat the ID DS such that the septum of the IDDS faces the syringe, wherein the top bore portion and the bottom bore portion are spaced apart from each other such that the needle of the syringe extends into the IDDS.

In addition to one or more aspects of the first embodiment of the filling system, or as an alternative, the wall of the filling fixture defines an axial slot extending from the top to the bottom of the filling fixture to provide visual access to the passage.

In addition to one or more aspects of the first embodiment of the filling system, or as an alternative, the filling system includes a sheath extending from a top end to a bottom end and having a cylindrical shape, wherein the sheath is configured to slide over the needle of the syringe so that the top end of the sheath is against the bottom of the barrel, whereby the sheath surrounds a portion of the needle, to prevent bending of needle, and wherein the needle is configured to extend past the bottom end of the sheath to engage the IDDS.

In addition to one or more aspects of the first embodiment of the filling system, or as an alternative, the filling system includes a centering cone having an outer cone surface defining an outer cone shape with wide end and a narrow end, and the centering cone defines a center passage such that the centering cone is configured for being positioned around the needle of the syringe; wherein: the centering cone has a cone length such that when the wide end is positioned against the bottom of the barrel of the syringe, the needle is configured to extend past the narrow end of the cone to engage the IDDS; and the passage of the filling fixture has a conical bore portion with a conical shape between the top bore portion and the bottom bore portion that is complementary to the outer cone shape of the centering cone, such that when the IDDS is seated within the syringe and the centering cone is disposed against the syringe, the conical bore portion is configured to guide the centering cone into the filling fixture, and thereby guide the needle into the IDDS.

Disclosed is a method filling an implantable drug delivery system (IDDS) with a syringe having a drug, wherein the syringe has a barrel extending from a barrel top to a barrel bottom, a plunger extending from the barrel top and a needle extending from the barrel bottom, and the IDDS defines a compartment with first and second ends that are opposite each other and a shell extending between the first and second ends, wherein: one of the first and second ends has a septum that is configured to receive the drug from the needle of the syringe; and one or more of another of the end first and second ends, and the shell, includes a porous surface, the method including: positioning the IDDS within a bottom bore portion of a passage that extends between a top and a bottom of a filling fixture such that the septum of the IDDS faces the top of the passage and the other end of the IDDS faces a drain passage defined at the bottom of the filling fixture; positioning the syringe in the filling fixture by sliding the syringe into a top bore portion at the top of the filling fixture, and into a guide portion of the passage, so that the syringe is guided into the filling fixture, whereby the needle engages the IDDS; and the method further includes injecting the drug via the plunger into the compartment of the IDDS.

In addition to one or more aspects of the method, or as an alternative, positioning the syringe in the filling fixture includes: sliding a sheath over the needle of the syringe so that a top end of the sheath is against the bottom of the barrel, whereby the sheath surrounds a portion of the needle, to prevent bending of needle, wherein the needle extends past the bottom end of the sheath.

In addition to one or more aspects of the method, or as an alternative, positioning the syringe in the filling fixture includes: sliding a centering cone over the needle such that a wide end of the centering cone is positioned against the bottom of the barrel of the syringe, and a narrow end of the centering cone engages the guide portion of the passage and guides the syringe into the filling fixture, and wherein the needle extends past a narrow end of the cone.

In addition to one or more aspects of the method, or as an alternative, the method includes viewing the injecting the drug via the plunger into the IDDS via an axial slot extending from the top to the bottom of the filling fixture, from an outer surface of the filling fixture and into the passage of the filling fixture.

Disclosed is at least another embodiment of a filling system that supports a syringe with a drug and an implantable drug delivery system (IDDS), wherein the syringe has a barrel extending from a barrel top to a barrel bottom, a plunger extending from the barrel top and a needle extending from the barrel bottom, and the IDDS defines a compartment with first and second ends that are opposite each other and a shell extending between the first and second ends, wherein: one of the first and second ends has a septum that is configured to receive the drug from the needle of the syringe; and one or more of another of the end first and second ends, and the shell, includes a porous surface, the filling system including a top member and a bottom member, wherein: the top member is configured to support the syringe, and the bottom member is configured to support the IDDS, such that the needle of the syringe faces the septum of the IDDS; and the top member is configured to move toward and away from the bottom member, to insert the needle into the IDDS via the septum of the IDDS, to thereby fill the compartment of the ID DS with the drug upon activation of the plunger.

In addition to one or more aspects of the another embodiment of the filling system, or as an alternative, the filling system includes a first post extending from the bottom member, through the top member, wherein the first post defines a first axis, and the top member is configured to move toward and away from the bottom member along the first axis, along the first post, to insert the needle into the IDDS via the septum of the IDDS.

In addition to one or more aspects of the another embodiment of the filling system, or as an alternative, the filling system includes a plurality of posts including the first post, extending from the bottom member, through the top member, wherein the posts are oriented parallel to each other, and spaced apart from each other within the system, and the top member is configured to move toward and away from the bottom member along the first axis, along the posts, to insert the needle into the IDDS via the septum of the IDDS.

In addition to one or more aspects of the another embodiment of the filling system, or as an alternative, the top member includes a top aperture that extends through the top member along an axis parallel to the first axis; the top aperture is configured to receive and seat the syringe such that the needle of the syringe extends toward the second member; and the bottom member defines a bottom aperture, wherein the bottom aperture extends parallel to the first axis to define a drain passage.

In addition to one or more aspects of the another embodiment of the filling system, or as an alternative, the filling system includes a first stop system, secured to the first post, that limits motion of the top member toward the bottom member and supports the top member relative to the bottom member.

In addition to one or more aspects of the another embodiment of the filling system, or as an alternative, the first stop system includes: a wedge, secured to the first post via a wedge bracket, having a wedge top and a wedge bottom, wherein: the wedge top engages the top member of the filling system when the top member is moved toward the bottom member, to thereby limit motion of the top member toward the bottom member and support the top member relative to the bottom member.

In addition to one or more aspects of the another embodiment of the filling system, or as an alternative, the filling system includes a syringe adapter configured for being removably inserted into the top aperture of the top member, wherein: the syringe adapter defines a cup-shape with an adapter cavity that is sized to receive and seat the barrel of the syringe; and the needle of the syringe extends through the bottom portion of the syringe adapter when the barrel of the syringe is seated within the syringe adapter.

In addition to one or more aspects of the another embodiment of the filling system, or as an alternative, the filling system includes a bottom fixture defining a fixture center aperture that extends parallel to the first axis, wherein the fixture aperture is sized to receive the IDDS, and wherein the bottom fixture is configured for being fixed against the bottom member so that the fixture center aperture is aligned with the bottom aperture.

In addition to one or more aspects of the another embodiment of the filling system, or as an alternative, the plurality of posts includes a marker post, wherein a top portion of the marker post includes position markers for identifying a position of the top member relative to the bottom member when the top member is moved relative to the bottom member to insert the needle into the IDDS via the septum of the IDDS.

In addition to one or more aspects of the another embodiment of the filling system, or as an alternative, the top aperture of the top member defines: a top portion that is sized to receive and seat the barrel of the syringe; and a bottom portion that is narrower than the top portion of the top aperture and sized for the needle of the syringe to extend through the top member when the barrel of the syringe is seated in the top portion of the top aperture.

In addition to one or more aspects of the another embodiment of the filling system, or as an alternative, the bottom aperture of the bottom member defines: a top portion, with a top edge defining a top chamfer; and a bottom portion of that is larger than the top portion of the bottom aperture, whereby the top edge defines a seat for seating a portion of the IDDS within the bottom aperture of the bottom member, thereby securely positioning the IDDS within the filling system.

In addition to one or more aspects of the another embodiment of the filling system, or as an alternative, the first stop system includes: a biasing member extending upwardly from the bottom member toward the top member; and a clamp secured to the first post to react biasing motion from the biasing member, and thereby lock the first member against the first post.

The above described and other features are exemplified by the following figures and detailed description.

Any combination or permutation of embodiments is envisioned. Additional advantageous features, functions and applications of the disclosed assemblies, methods and devices of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

The following figures are exemplary embodiments wherein the like elements are numbered alike.

Features and aspects of embodiments are described below with reference to the accompanying drawings, in which elements are not necessarily depicted to scale.

Exemplary embodiments are further described with reference to the appended figures. It is to be noted that the various features, steps, and combinations of features/steps described below and illustrated in the figures can be arranged and organized differently to result in embodiments which are still within the scope of the present disclosure. To assist those of ordinary skill in the art in making and using the disclosed assemblies, methods and devices, reference is made to the appended figures, wherein:

FIG. 1 is a side cross-sectional view of an exemplary drug delivery system, according to the present disclosure.

FIG. 2 is a modeled delivery profile of the drug delivery system of FIG. 1 and for an exemplary drug.

FIG. 3 is a modeled release profile of drug showing percent released over time for an exemplary drug delivery system according to the present disclosure.

FIG. 4 is a side cross-sectional view of another exemplary drug delivery system, according to the present disclosure.

FIG. 5 is a side view of another exemplary drug delivery system, according to the present disclosure.

FIG. 6 is an exploded partial view of the drug delivery system of FIG. 5.

FIG. 7 is an exploded side view of the drug delivery system of FIG. 5.

FIG. 8 is a side perspective view of the drug delivery system of FIG. 5.

FIG. 9 is a cross-sectional side view of another exemplary drug delivery system, according to the present disclosure.

FIG. 10 is a side perspective view of another exemplary drug delivery system, according to the present disclosure.

FIG. 11 is an exploded side view of the drug delivery system of FIG. 10, and showing the bottom port area in a side cross-sectional view.

FIG. 12 is a cross-sectional side view of another exemplary drug delivery system, according to the present disclosure.

FIG. 13 is a cross-sectional schematic view of a drug filling system for filling a drug delivery system, and showing a syringe with a drug and the drug delivery system filled by the syringe, according to the present disclosure.

FIG. 14 is a perspective view of the drug filling system of FIG. 13, according to the present disclosure.

FIG. 15 is an end view of the drug filling system of FIG. 13, along view-line 15-15 shown in FIG. 13, according to the present disclosure.

FIG. 16 is an end schematic view of another embodiment of a drug filling system for filling a drug delivery system, according to the present disclosure.

FIG. 17 is a cross-sectional schematic view of the drug filling system of FIG. 16, and showing a syringe with a drug and the drug delivery system filled by the syringe, according to the present disclosure.

FIG. 18 is a top perspective view of a portion of the drug filling system of FIG. 16, according to the present disclosure.

FIG. 19 is a bottom perspective view of a portion of the drug filling system of FIG. 16, according to the present disclosure.

FIG. 20 is a detailed view of a portion of the drug filling system identified in FIG. 18, and showing the drug delivery system, according to the present disclosure.

FIG. 21 is an exploded view of another embodiment of a drug filling system for filling a drug delivery system, and showing a syringe utilized with the drug filling system, according to the present disclosure, where the drug filling system includes a sheath, a centering cone and a fixture.

FIG. 22 is a view of the syringe with the sheath and the centering cone of the drug filling system of FIG. 21 installed on the syringe, according to the present disclosure.

FIG. 23 is a view of the syringe with a sheath and centering cone of the drug filling system of FIG. 21 installed on the syringe, according to the present disclosure.

FIG. 24 is a cross-sectional view of the drug filling system of FIG. 21 installed on the syringe, and showing the drug delivery system within the fixture, according to the present disclosure.

FIG. 25 is a flowchart shows a method filling the IDDS with the syringe having the drug, according to the present disclosure.

### DETAILED DESCRIPTION OF THE DISCLOSURE

The exemplary embodiments disclosed herein are illustrative of advantageous drug delivery systems, or more specifically implantable drug delivery systems (or IDDS) and methods/techniques related to the same. It should be understood, however, that the disclosed embodiments are merely exemplary of the present disclosure, which may be embodied in various forms. Therefore, details disclosed herein with reference to exemplary drug delivery systems and associated processes/techniques of assembly and use are not to be interpreted as limiting, but merely as the basis for teaching one skilled in the art how to make and use the advantageous drug delivery systems and/or alternative drug delivery systems of the present disclosure.

Disclosed herein are advantageous drug delivery systems, and related methods of fabrication and use thereof.

The present disclosure provides improved drug delivery systems for extended drug delivery (e.g., via passive diffusion) and/or provides tunability, and improved systems/methods for utilizing and fabricating the drug delivery systems.

More particularly, the present disclosure provides single or dual compartment, and single or dual porous membrane based (e.g., porous zinc membrane based) drug delivery systems for extended drug delivery (e.g., via passive diffusion) and/or to provide tunability, i.e., drug delivery where the release of the drug can be adjusted to achieve a desired regimen.

Referring now to the drawings, like parts are marked throughout the specification and drawings with the same reference numerals, respectively. The figures are not necessarily to scale, and, for example, in certain views the scale of the parts may have been exaggerated for purposes of clarity.

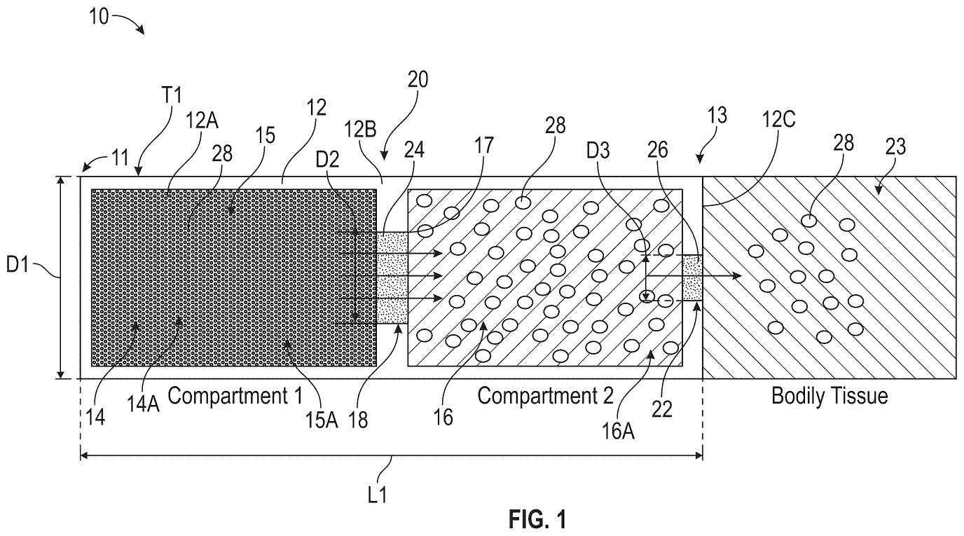

Turning to FIG. 1, there is illustrated a drug delivery system 10 depicting an embodiment of the present disclosure.

Exemplary drug delivery system 10 takes the form of a dual compartment and dual porous membrane based (e.g., porous zinc membrane based) drug delivery system 10 for extended delivery of a first drug 28 (for simplicity, a drug 28), e.g., via passive diffusion, and/or tunability or the like, although the present disclosure is not limited thereto.

As shown in FIG. 1, drug delivery system 10 includes a housing 12 that extends from a first end 11 to a second end 13. In exemplary embodiments, the housing 12 is substantially tubular or substantially cylindrical, although the present disclosure is not limited thereto. Rather, it is noted that the housing 12 can take a variety of shapes and/or forms.

The housing 12 can be fabricated from a variety of materials. For example, the housing 12 can be fabricated from a biocompatible metal (e.g., magnesium, zinc, titanium, iron, stainless steel, or an alloy thereof). The housing 12 can also be fabricated from a biocompatible polymer, e.g., a poly(meth)acrylate or copolymer thereof, a polyurethane, a polyether ketone, or the like; or a biodegradable polymer (e.g., a polyester such as a polycaprolactone, polylactide (e.g., poly(DL-lactide (PLA) or poly(L-lactide) (PLLA), or other isomers and copolymers of lactide), a polyglycolic acid, or a copolymer thereof, a polysaccharide, or the like. Where the housing 12 is biodegradable, it is preferably selected to have a slower biodegradability than any biodegradable substance associated with the drug 28, which is described below. In an embodiment, the housing 12 can be fabricated from a material such as zinc, iron, magnesium, titanium, metal alloys, polylactic acid, poly(lactic-co-glycolic acid), or a combination thereof. The outer wall (or outer shell) 12A of the housing 12 can be solid, or can be porous, or a combination of solid and porous, to allow the diffusion of the drug 28 as described below through the housing 12. In an embodiment, all of the outer wall 12A of the housing 12 is solid, except at portions defining the openings, generally referenced as openings 17. In an embodiment, if all or a portion of the outer wall 12A of the housing 12 is porous, it has a porosity that is less than a porosity of the porous membranes as described below.

In non-limiting examples, the housing 12 takes the form of a tube shape, with an overall length of about 0.5 to about 25 cm, preferably about 0.5 to about 10 cm, more preferably between about 1 to about 5 cm. The housing 12 can have a diameter D1 between about 1 to about 25 mm, preferably between about 2 to about 5 mm. The outer wall 12A of the housing 12 can have a wall thickness T1 between about 1 to about 5 mm. It is noted that the diameter D1 of the housing 12 can be between about 3 to about 7 mm, and the length L1 of the housing 12 can be between about 3 to about 12.5 mm.

An exemplary housing 12 defines a first compartment 14 and a second compartment 16 (each generally a compartment 15), with a first opening 18 in the housing 12 in fluid communication with the first and second compartments 14, 16. The first opening 18 is located in a separator wall (or plate) 12B located at a position 20 (e.g., an intermediate position 20) between the first and second ends 11, 13 of the housing 12. The shape of the first opening 18 may be circular in a non-limiting embodiment.

A second opening 22 in the housing 12 is positioned at the second end 13 of the housing 12 of the system 10, in an end wall (or plate) 12C, as shown in FIG. 1. The second opening 22 may have a circular cross section with a smaller area than the first opening 18, in a non-limiting embodiment. In general, the second opening 22 can be in fluid communication with an area 23 that is external to the housing 12 of the system 10 (e.g., an area 23 such as, for example, the surrounding tissue of a body of a patient, test subject, or the like, after the system 10 is positioned in the body). The patient can be a human or animal in need of the drug. The test subject can be a human or animal.

In exemplary embodiments, a first porous membrane 24 is positioned in the first opening 18, and a second porous membrane 26 is positioned in the second opening 22.

The first porous membrane 24 can be attached and/or bonded relative to the first opening 18 of the housing 12 and that the second porous membrane 26 can be attached and/or bonded relative to the second opening 18 of housing 12 via various attachment or bonding methods (e.g., sintering bonding, adhesive, press-fit, etc.).

As indicated above, FIG. 1 shows a cross section of housing 12 of the system 10 (e.g., tubular system 10), with the system 10 having first compartment 14 in fluid communication with second compartment 16 via first porous membrane 24 positioned in first opening 18, and with the system 10 having second compartment 16 in fluid communication with an area 23 of the surrounding tissue of a body of a patient via a second porous membrane 26 positioned in the second opening 22.

Each respective interior cavity 14A, 16A (each generally a cavity 15A), i.e., the interior space of the first and/or second compartments 14, 16, can be filled or partially filled with a drug 28, e.g., a solution including the drug 28 in dissolved or particulate form, a gel including the drug 28, a slow release composition including the drug 28, or the like, or a combination thereof. In an embodiment, the drug 28 is provided in dissolved or particulate form in a solution (e.g., water), and fills the compartment 15. When present as particles, the drug can be partially soluble, e.g., in water, and provide extended release of the dissolved drug over time. In other embodiments, the drug 28 is provided in other forms, such as reversibly attached to a gel or solid support (in dissolved or particulate form), present in or attached to a degradable (e.g., dissolvable) matrix such as a gel or solid support, encapsulated in one or more degradable shells, or a combination thereof. Such forms are known to those of skill in the art and are useful to provide even greater tunability of drug release. One or more adjuvants (e.g., salt, pH adjusting agent, or the like) as is known in the art can be present in addition to the drug.

In exemplary embodiments, the first porous membrane 24 is generally less porous, i.e., finer, having a smaller pore size relative to second porous membrane 26, although the present disclosure is not limited thereto. However, it is noted that the mean pore size of the first porous membrane 24 can be as large as about 20 μm, or as large as about 100 μm, or possibly even larger, for example between about 1 to about 500 μm.

The second porous membrane 26 is generally more porous, i.e., coarser, relative to the first porous membrane 24, with the second porous membrane 26 generally having a larger pore size than the first porous membrane 24, although the present disclosure is not limited thereto. An exemplary range of the mean pore size of the second porous membrane 26 can be between about 1 to about 100 μm, or between about 1 to about 500 μm, although the present disclosure is not limited thereto. In some embodiments, a mean pore size of the first porous membrane 24 can be substantially the same as or similar to the mean pore size of the second porous membrane 26.

In exemplary embodiments, the coarseness and microstructure of the second porous membrane 26 can prevent bio-fouling of system 10, whereas the fineness of the first porous membrane 24 can allow for precision dosage/control over diffusion/drug 28 delivery to eventual area 23.

As described above, it is also possible for all or a part of the outer wall 12A of the housing 12 to be porous. In an aspect, the outer wall 12A of the housing 12 can be solid at the first compartment 14 and porous at the second compartment 16. Alternatively, the outer wall 12A of the housing 12 can be porous at the first compartment 14 and solid at the second compartment 16. In an embodiment, the outer wall 12A of the housing 12 is fully porous. An exemplary range of the mean pore size of the housing 12 can be between about 1 to about 100 μm, or between about 1 to about 500 μm, and can vary, for example being finer in the outer wall 12A of the housing 12 forming the first compartment 14 and coarser in the outer wall 12A of the housing 12 defining the second compartment 16. In some embodiments, a mean pore size of the first porous membrane 24, the second porous membrane 26, and all or a portion of the housing 12 can be substantially the same as or similar to each other. In other exemplary embodiments, the coarseness and microstructure of the second porous membrane 26 and all or a portion of the housing 12 can prevent bio-fouling of system 10, whereas the fineness of the first porous membrane 24 can allow for precision dosage/control over diffusion/drug 28 delivery to eventual area 23.

In non-limiting examples, each porous membrane 24, 26 (and optionally all or a portion of the housing 12) that is utilized to regulate the mass transfer of the drug 28 can be fabricated from a biocompatible metal (e.g., magnesium, zinc, titanium, iron, stainless steel, or an alloy thereof). Each porous membrane 24, 26 can also be fabricated from a biocompatible polymer, e.g., a poly (meth) acrylate or copolymer thereof, a polyurethane, a polyether ketone, or the like; or a biodegradable polymer (e.g., a polyester such as a polycaprolactone, polylactide (e.g., PLA or PLLA, etc.), a polyglycolic acid, or a copolymer thereof, a polysaccharide, or the like. Where each porous membrane 24, 26 is biodegradable, each porous membrane 24, 26 is preferably selected to have a slower biodegradability than any biodegradable substance associated with the drug 28 or microprojections, which are described in further detail below. Each porous membrane 24, 26 can be in the shape of a flat cylinder or thin needle, with diameters between about 0.25 to about 10 mm and thicknesses between about 0.25 to about 10 mm, although the present disclosure is not limited thereto.

In general, each compartment 14, 16 is configured and dimensioned to house the drug 28 or active agent particles 28.

As such, FIG. 1 depicts an exemplary drug delivery system 10, with the system 10 having first and second compartments 14, 16, and having first and second porous membranes 24, 26, configured to regulate the delivery of a drug 28 to area 23 that is external to system 10.

The exemplary system 10 includes first and second compartments 14, 16 separated by the first porous membrane 24 in a separator plate 12B. The second compartment 16 has two openings 17, that is, a first opening 18 joining the first and second compartments 14, 16, and a second opening 22 that is in communication with area 23.

In exemplary embodiments, the system 10 is configured and dimensioned to be implanted in a body of a patient or the like (e.g., within the tissue of a body of a patient or the like). The system 10 can be located in other/different areas of the body or the like. M ore than one system 10 can be used, at different locations of the body.

The exemplary system 10 is an improvement over other conventional designs/assemblies, as the dual compartment 14, 16 feature of system 10 has been shown via modeling to be able to deliver the medication 28 within therapeutic concentrations over longer periods of time to area 23, and to be able to deliver a larger percentage of the loaded dose 28 within those same concentrations. The utilization of slow-or fast-release technologies within the compartment can provide even finer tuning of drug release. For example, a solution including the drug 28 can provide an initial faster (higher concentration) release for a first extended period of time, followed by a slower release over a second extended period of time as the drug is released within the compartment from, for example, a dissolvable gel or encapsulant. The utilization of a plurality of encapsulants, at 12A, for example, can provide multiple release concentrations over multiple extended time periods. For other therapies, where initial slow release (low concentration) is desired for a first extended period of time, followed by faster (higher concentration) over a second extended period of time can be provided, for example, by encapsulating the drug 28 separately at two concentrations, one lower and one higher, or an outer shell having a slower release rate and in inner shell having higher release rate that is exposed after erosion of the outer shell.

Some conventional approaches for extended drug release include an osmotic pump and some polymer matrix based compositions that deliver medication for an extended period of time up to thirty to 120 days (polymer matrix systems), and 180 to 360 days (osmotic pump systems). The passive diffusion based drug delivery of exemplary system 10 is able to deliver medication for 180 days or longer, for example, up to 1220 days. Moreover, this exemplary system 10 can increase the percent of the initial dose delivered while extending the time spent in a therapeutic concentration window while still maintaining a small footprint.

Furthermore, the second porous membrane 26 separating the second compartment 16 from the external area 23 can be much coarser than the first porous membrane 24 of system 10 separating the first and second compartments 14, 16. This can allow for the retention of precise drug delivery with the finer (first) media of membrane 24, and can prevent bio-fouling as the coarser (second) porous membrane 26 is inherently resistant, as determined by testing, to the formation of protein films (e.g., on the second end 13 of the housing 12). In some embodiments, the first porous membrane 24 is coarser (more permeable) than the second porous membrane 26, depending, e.g., on how quickly the drug is intended to reach the patient. A rapid dispensing to a patient may be desired if the drug in the second compartment is not stably stored there, due to the nature of the applied excipient in that compartment. For example, if the drug were stably stored in the first compartment, due to the excipient in that compartment, it may be stored there for an extended period of time, depending on the membrane utilized between the compartments. Then, as the drug diffuses to the second compartment, there may be a desire to have it diffuse to the patient more rapidly because of the different excipient utilized in the second compartment. For that reason, a more permeable membrane may be placed at the outlet of the assembly. It is to be appreciated that various techniques can provide the greater or lesser permeability in the different porous members, and coarseness is one of several nonlimiting options in obtaining the desired permeability.

In an example embodiment, the internal volume of the housing 12 can be about 1 mL, with the internal volume divided into a 0.6 mL compartment defined by the first compartment 14, and with a 0.4 mL compartment defined by the second compartment 16. It is noted that a total internal volume of the first compartment 14 and of the second compartment 16 (added together) can range from about 0.10 mL to 5 mL, preferably between 0.10 to 2 mL.

The overall diameter D1 of the housing 12 of system 10 can be about 7 mm, and the length L1 of the housing 12 of the system 10 can be about 4 cm. The utilization of extended drug delivery mechanisms as described above, in particular dissolvable gels or a solid support, can result in larger dimensions.

In an example embodiment, the second porous membrane 26 can be classified as a Media Grade (“MG”) 0.1 having a mean pore size of about 0.1 μm. The diameter D3 of this exemplary second porous membrane 26 can be about 0.5 mm by about 1 mm thick. The first porous membrane 24 can be classified as a MG 2, with a diameter D2 of about 1.5 mm by about 3 mm thick. The first and second membranes 24, 26 can be fabricated out of 99.99% pure zinc, although the present disclosure is not limited thereto. It is to be appreciated that the diameter is another variable in controlling the mass transport rate of the drug through the membrane. Thus, the relative sizes of the diameters of the membranes as shown and discussed is not intended on limiting the scope of the embodiments.

In use, exemplary system 10 and the other embodiments described herein can be utilized for extended delivery of the drug 28, which includes any therapeutic or active agent such as a medication, pharmaceutical, supplement, or the like, or a reporting compound (e.g., a radiotracer or fluorescent compound) for monitoring drug delivery or other biological process. The drug can be a biologic, a protein, a pharmaceutical or reporter small molecules (10 to 1,000 g/mol), or a combination thereof. In an embodiment, the drug 28 is a biologic. Preferably, the sustained release of the drug 28 is for a period of greater than six months, for example, more than 6 months to two years or more than 6 months to one year. This can overcome difficulties with daily, weekly, or monthly dosing regimens.

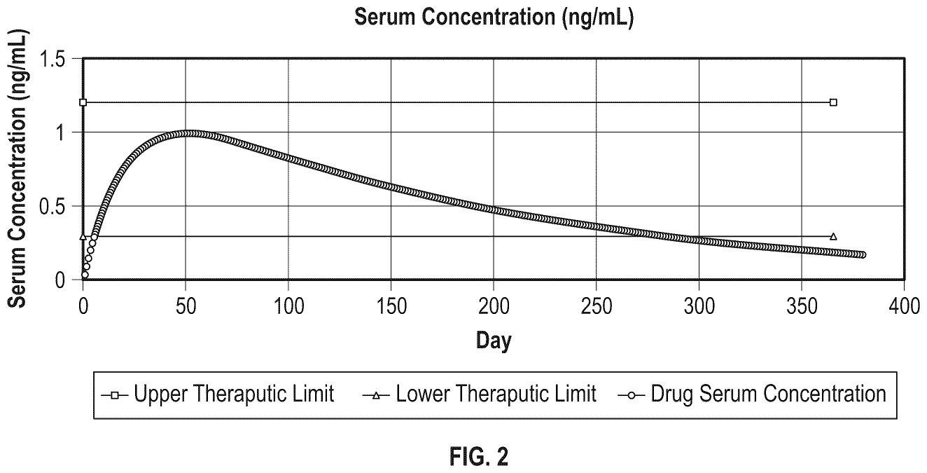

In FIG. 2, the release profile of a drug 28 through an exemplary system 10 (FIG. 1) is shown. For this example, the drug 28 was only loaded in the first compartment 14 (behind the first membrane 24), but the drug 28 could be loaded in both compartments 14, 16 theoretically. In FIG. 2, the y-axis details serum concentration, or a description of how concentrated the drug 28 is predicted to be in the blood after x days. The orange and gray lines detail a concentration window where the serum concentration indicates that the drug 28 is effective. The exemplary system 10 having both compartments 14, 16 allows for an extension of the time spent in this window of drug effectiveness by better regulating the diffusion gradient between the second compartment 16 and the area 23 by introducing an intermediate volume (compartment 16) and membrane 24 to facilitate mass transfer.

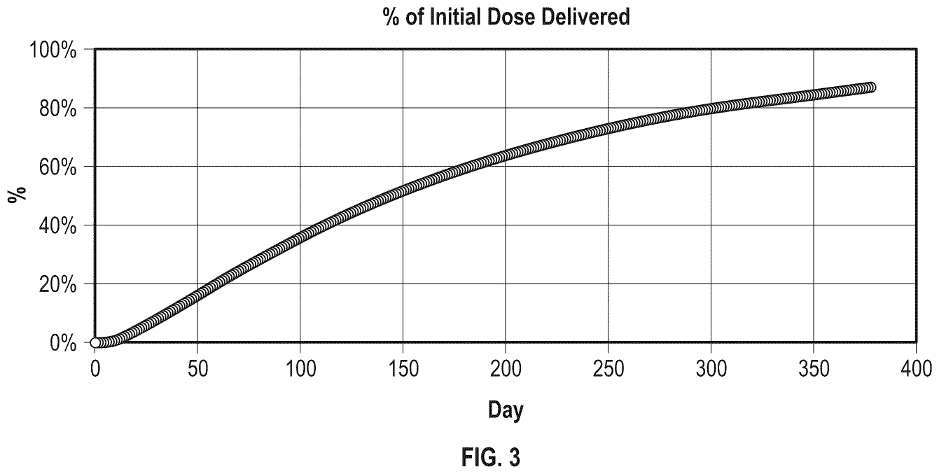

In FIG. 3, the predicted percent released over time relationship for an system 10 (FIG. 1) having compartments 14, 16 is shown. A goal of a drug delivery system 10 is to have the percent released close to 90% or greater at the time which the serum concentration is modeled to be below the minimum effective therapeutic level. An system 10 having both compartments 14, 16, via a better control over the drug delivery physics described above, allows for the achievement of a higher percentage delivered at this time.

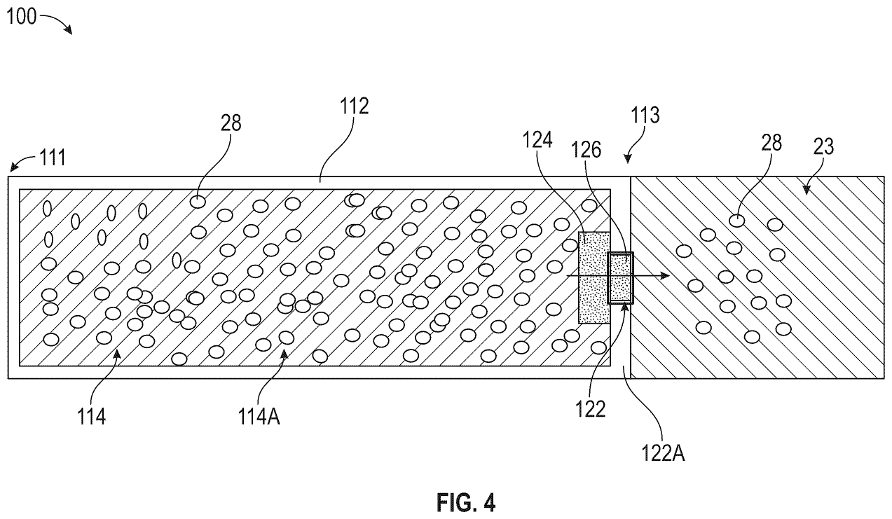

In another embodiment and as shown in FIG. 4, exemplary drug delivery system 100 takes the form of a single compartment 114 and dual porous membrane 124, 126 based (e.g., porous zinc membrane based) drug delivery system 100 for extended drug 28 delivery (e.g., via passive diffusion) and/or tunability or the like.

The exemplary drug delivery system 100 includes a housing 112 that extends from a first end 111 to a second end 113. In exemplary embodiments, the housing 112 is substantially tubular or substantially cylindrical, although the present disclosure is not limited thereto. Rather, it is noted that the housing 112 can take a variety of shapes and/or forms.

The housing 112 can be fabricated from a variety of materials as described above. For example, the housing 112 can be fabricated from a metal (e.g., magnesium, zinc, iron, stainless steel, or an alloy thereof). The housing 112 can also be fabricated from a biodegradable plastic (e.g., PLA or PLLA, etc.), a polyglycolide, a polysaccharide, or the like.

In non-limiting examples, the housing 112 takes the form of a tube shape, as similarly discussed above relative to housing 12 of system 10.

The exemplary housing 112 defines a compartment 114. An opening 122 in an end wall 122A of the housing 112 is positioned at the second end 113 of the housing 112 of the system 100, as shown in FIG. 4. The opening 122 may have a circular shape in a non-limiting embodiment. In general, the opening 122 can be in fluid communication with an area 23 that is external to the housing 112 of system 100 (e.g., an area 23 such as, for example, within the surrounding tissue of a body of a patient, or test subject, after system 100 is positioned in the patient).

In exemplary embodiments, a first porous membrane 124 is positioned within the compartment 114, proximal to the opening 122, and a second porous membrane 126 is positioned substantially within the opening 122, as discussed further below.

The first porous membrane 124 can be attached and/or bonded relative to the opening 122 of housing 112 (and/or to membrane 126) and that the second porous membrane 126 can be attached and/or bonded relative to the opening 122 of housing 112 (and/or to membrane 124) via various attachment or bonding methods (e.g., sintering bonding, adhesive, press-fit, etc.).

An interior cavity 114A defined by the compartment 114 can be filled with a drug 28 as described above.

In exemplary embodiments, the first porous membrane 124 is generally finer, having a smaller pore size relative to the second porous membrane 126. In certain embodiments, the first porous membrane 124 has a smaller mean pore size than the second porous membrane 126.

An exemplary range of the mean pore size of the first porous membrane 124 can be between about 0.05 to about 1.0 μm. The second porous membrane 126 is generally coarser relative to the first porous membrane 124, with the second porous membrane 126 generally having a larger pore size than the first porous membrane 124.

An exemplary range of the mean pore size of the second porous membrane 126 can be between about 1 to about 100 μm, although the present disclosure is not limited thereto.

In exemplary embodiments, these two membranes 124, 126, are joined or attached together to form one continuous body, leaving a porous matrix 124 and 126 with a gradient pore size structure. In an embodiment, the two membranes are a single membrane having a porous gradient (stepwise or continuous) of coarser to finer porosities from one surface to the opposite surface.

In exemplary embodiments, the coarseness (larger pores) of the second porous membrane 126 can prevent bio-fouling of system 100, and the tightness of the first porous membrane 124 can allow for precision dosage/control over diffusion/drug delivery to the eventual area 23.

In non-limiting examples, each porous membrane 124, 126 utilized to regulate the mass transfer of the drug 28 can be fabricated from zinc, titanium, polyether ether ketone, or another applicable or suitable biomaterial as described for the porous membranes 24, 26. Each porous membrane 124, 126 can be in the shape of a flat cylinder or thin needle, with diameters between about 0.25 to about 5 mm and thicknesses between about 0.25 to about 10 mm, although the present disclosure is not limited thereto.

In general, the compartment 114 is configured and dimensioned to house the drug 28, e.g., whether in solution, as particles, or other form as described above.

As such, FIG. 4 depicts an exemplary drug delivery system 100, with the system 100 having the compartment 114, and having first and second porous membranes 124, 126, configured to regulate the delivery of the drug 28 to the area 23 that is external to the system 100.

In exemplary embodiments, it is noted that the system 100 is configured and dimensioned to be implanted in a body of a patient, test subject, or the like (e.g., within the tissue of a body of a patient, test subject, or the like). It is also noted that the system 100 can be located in other/different areas of the body or the like, as described above. More than one system 100 can be used in different locations of the body.

In use, the exemplary system 100 can be utilized for the extended (sustained) delivery of the drug 28. Preferably, the sustained release of the drug 28 is for a period of greater than six months, for example, more than 6 months to two years or more than 6 months to one year. This can overcome difficulties with daily, weekly, or monthly dosing regimens.

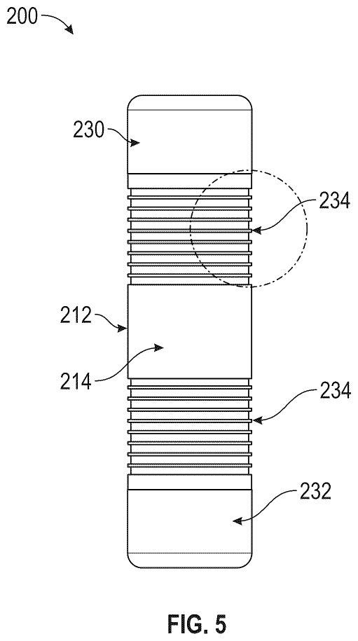

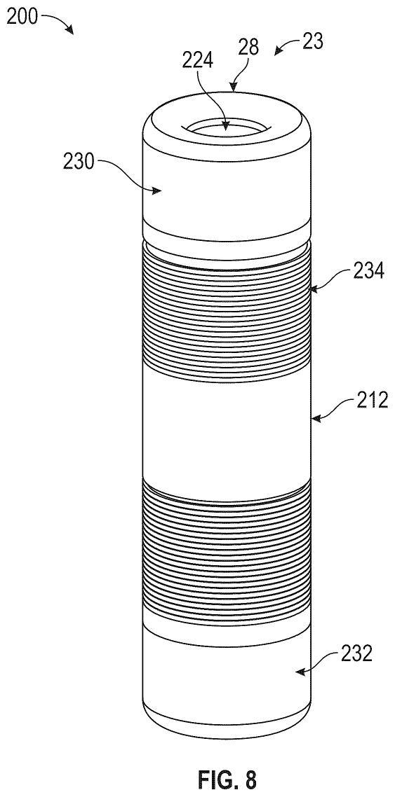

With reference to one or more of FIGS. 5-12, other embodiments of the exemplary drug delivery system 200 includes a housing 212 that extends from a first end 211 to a second end 213. In exemplary embodiments, the housing 212 is substantially tubular or substantially cylindrical with a hollow interior, although the present disclosure is not limited thereto. Rather, it is noted that the housing 212 can take a variety of shapes and/or forms.

It is noted that the housing 212 can be fabricated from a variety of materials as described above. For example, the housing 212 can be fabricated from a metal (e.g., magnesium, zinc, titanium, iron, stainless steel, or an alloy thereof). The housing 212 can also be fabricated from a biodegradable polymer (e.g., PLA or PLLA, etc.).

In non-limiting examples, the housing 212 takes the form of a tube or substantially cylindrical shape, as similarly discussed above. In exemplary embodiments, the housing 212 can extend about 1.02 cm or about 1.29 cm or about 1.63 cm from first end 211 to second end 213. In exemplary embodiments, the housing 212 can have a wall thickness of about 0.1016 cm.

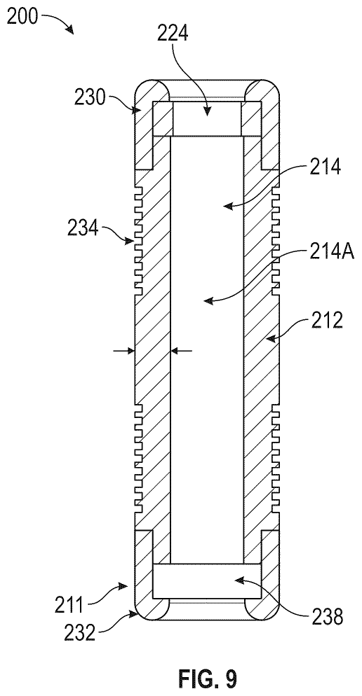

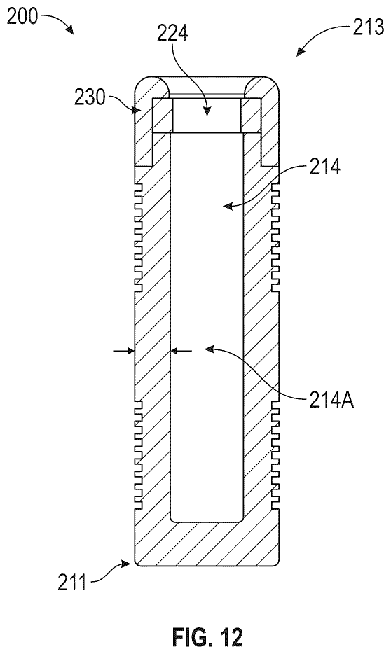

The exemplary housing 212 defines a compartment 214 shown explicitly in FIGS. 9 and 12. The interior cavity 214A of the compartment 214 can be fully or partially filled with a drug 28 as discussed above.

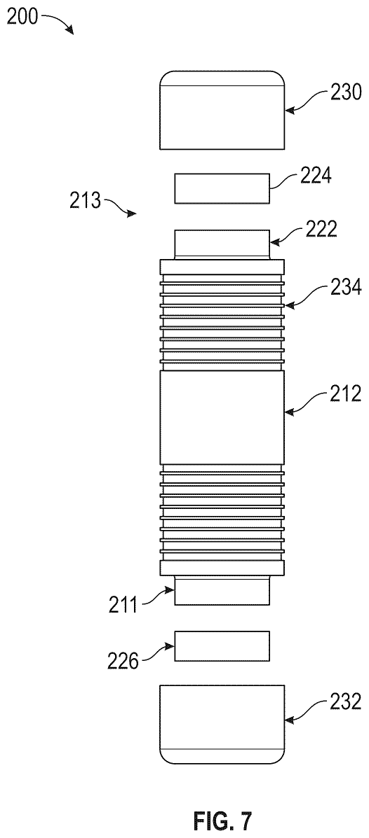

In certain embodiments, a first porous membrane 224 is positioned proximal to the opening 222, and a second porous membrane 226 is positioned proximal to first end 211 as shown in FIG. 7, or distant from first end 211, as discussed further below.

An opening 222 in the housing 212 is positioned at the second end 213 of housing 212 of the implantable drug delivery system 200 (otherwise referred to as an assembly or device). In general, opening 222 can be in communication with an area that is external to the housing 212 of the system 200 (e.g., an area such as, for example, within the surrounding tissue of a body of a patient or test subject), after the system 200 is positioned in the body. In exemplary and non-limiting embodiments, the compartment can have a total internal volume of about 0.25 ml or about 0.50 ml or about 1.00 ml.

In some embodiments, the first porous membrane 224 can be attached and/or secured relative to the opening 222 of the housing 212 via a first end cap 230 attached and/or secured to the second end 213, and the second porous membrane 226 (if adjacent to the first end 211) can be attached and/or secured relative to the first end 211 of the housing 212 via a second end cap 232 attached and/or secured to the first end 211. It is noted that the first end cap 230 can include a hood-like feature that is designed to prevent fibrosis from impeding the diffusion properties of the first porous membrane 224. In exemplary embodiments, the first end cap 230 can have an outer diameter of about 0.34 cm or about 0.43 cm or about 0.54 cm.

An exemplary range of the mean pore size of the first and second porous membranes 224, 226 can each be between about 0.10 to about 100 μm, although the present disclosure is not limited thereto.

In exemplary embodiments, each porous membrane 224, 226 (utilized to regulate the mass transfer of the drug 28) can be fabricated from a material as described above, for example, from zinc, titanium, polyether ether ketone, or another applicable or suitable biomaterial. Each porous membrane 224, 226 can be in the shape of a flat cylinder or thin needle, with diameters between about 0.25 to about 10 mm and thicknesses between about 0.25 to about 10 mm, although the present disclosure is not limited thereto.

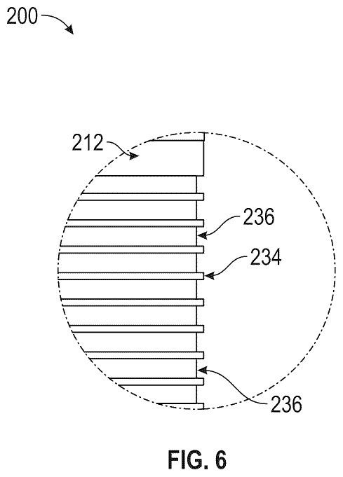

Further with reference to FIGS. 5-12, the housing 211 includes at least one grooved or threaded section (or threaded feature) 234 (e.g., two or more sections 234). Such grooved or threaded section can optionally be present in any of the embodiments discussed herein. Each grooved or threaded section 234 includes a plurality of tissue grooves 236 (see, e.g., FIG. 6). As such, the outside surface of the housing 212 is lined with grooved or threaded sections 234 having a plurality of tissue grooves 236, with the tissue grooves 236 promoting the adhesion of tissue to the system 200 (e.g., to prevent system 200 migration within the body). The grooved or threaded sections 234 modify the surface roughness of the exterior of the system 200 to promote tissue growth around the system 200 to hold it in place. As such, the grooved or threaded sections 234 along the housing 212 act to promote tissue adhesion and reduce implant system 200 migration. In an embodiment, the thread sections 234 conforms to a #6-40 thread classified by ASME B1.1.

Features such as hooks/loops can be added to the housing 212 (or the housing 112) to promote suturing of the system 200 (e.g., to a piece of the dermal layer). Additionally, polymeric coatings can be applied to the housing 212 (or the housing 114) to modify the chemical and/or physical properties at the surface of the housing 212 of the system 200. It is noted that a large range of surface roughening processes, such as various types of threading, sanding, chemical etching, sand blasting, and other modification methods can also be utilized on the housing 212 (or the housing 112) to promote the adhesion of tissue to the system 200. Preferably, raised surface features in the range of 200 to 500 microns can be created on the surface of the housing 212 to promote adhesion of tissue to the system 200.

It is noted that an entirely dissolvable design of the housing 112 (or the housing 212) can be fabricated from zinc, and a refillable design of the housing 112 (or the housing 212) can be fabricated from titanium, although the present disclosure is not limited thereto.

FIGS. 5-8 depict an embodiment of an exemplary drug delivery system 200, with the system 200 defining a first compartment having first and second porous membranes 224, 226 to regulate the delivery of a drug 28 to area 23 that is external to system 200.

In an embodiment, the second porous membrane 226 can be attached to a filling apparatus or the like in order to fill the compartment 214 with the drug 28, either before or after the system 200 is implanted. The second porous membrane 226 can be closed after filling the compartment 214 with the drug 28 (e.g., closed with a plug of hydroxyapatite cement or other biocompatible cement).

FIG. 9 depicts another exemplary drug delivery system 200, with the system 200 having the compartment 214, and having first porous membrane 224 secured to housing 212 via first end cap 230 at the second end 213, and with a septum member 238 secured to first end 211 via the second end cap 232. Though the porous member 224 is identified as being located at the second end 213 and the septum member 238 is identified as being located at the first end 211, this is not intended on limiting the scope of the embodiments. Generally, one of the first and second ends 211, 213, which are separated along the shell 12A, may have the septum 238 while the other of the first and second ends 211, 213 may have the porous member 223. In some embodiments, one of the first and second ends 211, 213 may have the septum 238 while one or more of the other of the first and second ends 211, 213 and the shell 12A (FIG. 1) extending between the first and second ends may have or be defined at least in part by (i.e., may include) a porous surface, which may be the porous member 224.

The septum member 238 can be attached to a filling apparatus or the like in order to fill compartment 214 with drug 28, either before or after system 200 is implanted. The septum member 238 can be closed, if desired, after filling compartment 214 with the drug 28 (e.g., closed with a plug of hydroxyapatite cement or other biocompatible cement, or a solid cap 232 having no opening therein).

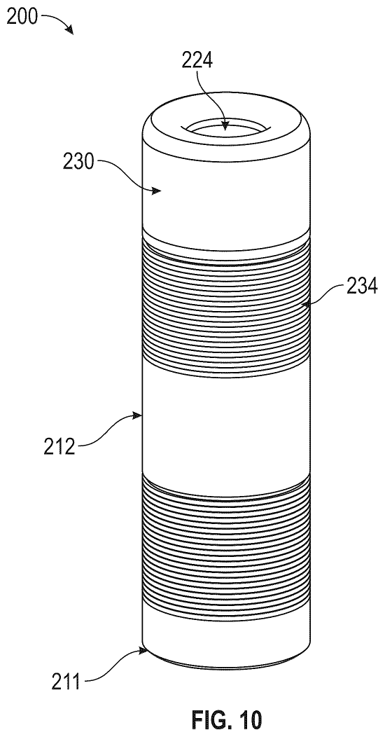

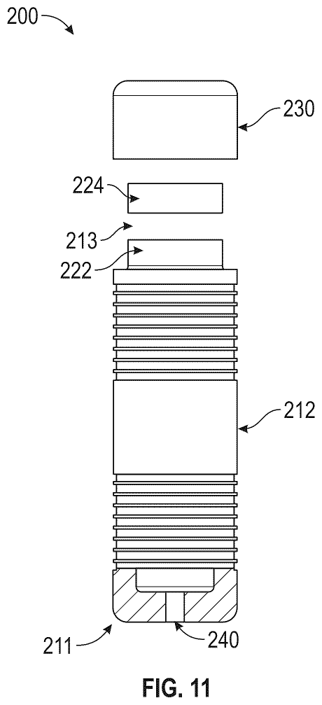

FIGS. 10-11 depict an exemplary drug delivery system 200, with the system 200 having compartment 214, and having first porous membrane 224 secured to the housing 212 via the first end cap 230, and with a drug loading port 240 positioned at the first end 211. The drug loading port 240 can be attached to a filling apparatus or the like in order to fill the compartment 214 with the drug 28 or active agent particles 28, either before or after system 200 is implanted. The drug loading port 240 can be closed, if desired, after filling the compartment 214 with the drug 28 (e.g., closed with a hydroxyapatite cement or other biocompatible cement).

FIG. 12 depicts an exemplary drug delivery system 200, with the system 200 having the compartment 214, and having the first porous membrane 224 secured to the housing 212 via the first end cap 230, and with the first end 211 being closed off. The compartment 214 can be filled with the drug 28 or active agent particles 28 via the second end 213 (e.g., before the membrane 224 is secured to the housing 212).

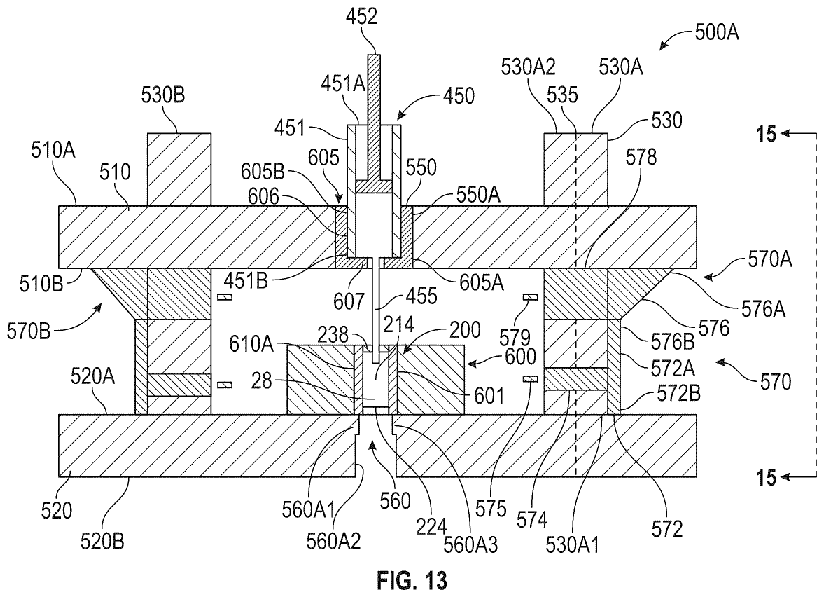

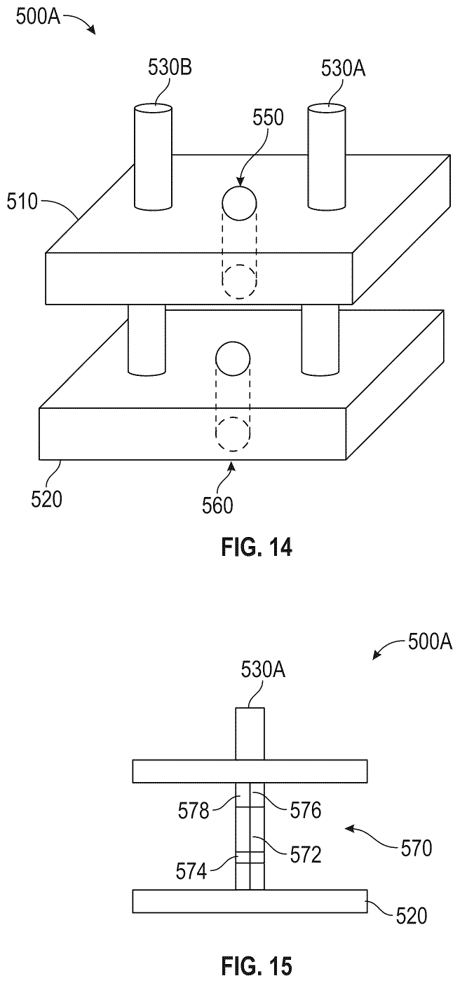

Turning to FIGS. 13-15, a first embodiment of a filling system (or drug filling system) 500A is shown. Each embodiment of the filling systems disclosed herein may generically be referred to as a filling system 500. The filling system 500A is generally shown as an H-frame configuration. The filling system 500A supports a syringe 450 with a drug 28 and an implantable drug delivery system (IDDS) 200. Though the IDDS 200 is identified in these and later figures, any IDDS disclosed herein may be utilized with the disclosed filling systems 500.

The syringe 450 has a barrel 451 extending from a barrel top 451A to a barrel bottom 451B, a plunger 452 extending from the barrel top 451A and a needle 455 extending from the barrel bottom 451B. The IDDS 200 defines a compartment 214 with a first end having a porous membrane 224 and a second end having a septum 238 that is configured to receive a drug 28 from the needle 455 of the syringe 450.

The filling system 500A includes a top member 510 and a bottom member 520. The top and bottom members 510, 520 may be plates and more specifically rectangular or square plates, as nonlimiting examples, with respective top surfaces 510A, 520A and bottom surfaces 510B, 520B.

The top member 510 is configured to support the syringe 450. The bottom member 520 is configured to support the IDDS 200, such that the needle 455 of the syringe 450 faces the septum 238 of the IDDS 200. The top member 510 is configured to move toward and away from the bottom member 520, to insert the needle 455 into the IDDS 200 via the septum 238 of the ID DS 200. From this configuration, the compartment 214 of the IDDS 200 may be filled with the drug 28 upon activation of the plunger 452.

A first post 530A (or generally a post 530) extends from a bottom 530A1 to a top 530A2. The bottom 530A1 of the first post 530A is located at the bottom member 520, and may be fastened to the bottom member 520. The first post 530A extends through the top member 510 so the top 530A2 of the first post 530A is above the top member 510. The first post 530A defines a first axis 535. The top member 510 is configured to move toward and away from the bottom member 520 along the first axis 535, to insert the needle 455 into the IDDS 200 via the septum 238 of the IDDS 200.

More specifically, the system 500A includes plurality of posts 530, e.g. first and second posts 530A, 530B, extending from the bottom member 520, through the top member 510. The posts 530 may have a same shape and size as each other, as a non-limiting embodiment. The posts 530 may be oriented parallel to each other, and spaced apart from each other within the system 500A. From this configuration, the top member 510 is configured to move toward and away from the bottom member 520 along the first axis 535, via the posts 530, to insert the needle 455 into the IDDS 200 via the septum 238 of the IDDS 200.

The top member 510 includes a top aperture (or first channel) 550, defined by a top aperture wall 550A that may be cylindrically shaped. The top aperture 550 extends through the top member 510 parallel to the first axis 535. The top aperture 550 is configured to receive and fixedly seat the syringe 450 such that the needle 455 of the syringe 450 extends toward the second member 520.

The bottom member 520 has a bottom aperture 560 (or second channel), defined by a bottom aperture wall 560A that may be cylindrically shaped. The bottom aperture 560 extends parallel to the first axis 535. The bottom aperture 560 defines a drain passage or port for wet-out effluent.

A first stop system 570A (or generally a stop system 570) is secured to the first post 530A. The first stop system 570A limits motion of the top member 510 toward the bottom member 520. The first stop system 570A also supports the top member 510 relative to the bottom member 520. As shown, the system 500A includes a plurality of stop systems 570 including the first stop system 570A and a second stop system 570B that is secured to the second post 530B. The first and second stop systems 570A, 570B may be the same as each other so that the discussion herein shall focus on the first stop system 570A.

A syringe adapter (or needle placement fixture) 605 is configured for being removably inserted into the top aperture 550 of the top member 510. The syringe adapter 605 has an adapter outer wall 605A that may be cylindrically shaped. The syringe adapter 605 is sized to fit within the top aperture 550, and the fit may be a friction fit as a nonlimiting example. The syringe adapter 605 is cup-shaped with an adapter cavity 606 defined by an adapter inner wall 605B. The adaptor cavity 606 is sized to receive the barrel 451 of the syringe 450, and the fit may be a friction fit as a nonlimiting example. The syringe adapter 605 defines a bottom portion 607 for seating the barrel bottom 451 of the syringe 450. The needle 455 of the syringe 450 extends through the bottom portion 607 of the syringe adapter 605 when the barrel 451 of the syringe 450 is seated within the syringe adapter 605.

A bottom fixture 600 (otherwise referred to as alignment fixture block, or IDDS placement fixture) has a fixture center aperture 601, defined by a fixture aperture wall 601A that may be cylindrically shaped. The fixture aperture 601 extends parallel to the first axis 535. The fixture aperture 601 is sized to receive the IDDS 200, and the fit may be a friction fit as a nonlimiting example. The bottom fixture 600 is configured for being fixed against the bottom member 520, i.e., against the top surface 520A of the bottom member 520, so that the fixture center aperture 601 is aligned with the bottom aperture 560.

The bottom aperture 560 of the bottom member 520 has a top portion 560A1 and a bottom portion 560A2. The top portion 560A1 of the bottom aperture 560 is narrower, e.g., in diameter, than the bottom portion 560A2 to define a shoulder or rim 560A3. With this configuration, the bottom member 510 defines a seat for seating the IDDS 200 at the top surface 520A of the bottom member 520 when the bottom fixture 600 is against the top surface 520A of the bottom member 520.

The first stop system 570A includes a runner 572 (or support plate). The runner 572 has a runner top 572A and a runner bottom 572B. The runner 572 is secured to the first post 570 via a runner bracket 574 (or bottom bracket). The runner bracket 574 may be secured with a bottom fastener 575. The runner 572 extends upwardly from the bottom member 520 toward the top member 510.

The first stop system 570A includes a wedge 576 (or stop wedge), secured to the first post 540 via a wedge bracket 578 (or top bracket). The wedge bracket 578 may be secured with a top fastener 579. The wedge 578 has a wedge top 578A and a wedge bottom 578B, which may be smaller than the wedge top 578A. With this configuration, the wedge 578 may distribute a supporting force over a wider area along the wedge top 578A. The wedge bottom 578B engages the runner top 572A and the wedge top 578A engages the bottom surface 510B of the top member 510 when the top member 510 is moved toward the bottom member 520. From this configuration, the first stop system 570A limits motion of the top member 510 toward the bottom member 520 and supports the top member 510 relative to the bottom member 520.

With the above embodiment, there is illustrated a first drug filling system 500A for filling any IDDS 10, 200 disclosed herein. The filling system includes a needle placement fixture 605 and an IDDS placement fixture 600. The frame 501, defined by the top and bottom members 510, 520 and posts 530, holds each fixture 600, 605, in moveable relation to each other. The system is configured to control placement of a needle 455 used to fill the IDDS 200. The needle 455 is accordingly in fluid communication with a source of a drug formulation, for example the syringe 450. The needle 45 can be of any gauge (“G”). However, the system 500A may be particularly useful with thin-gauge needles, for example, a 32G, 34G, 36G, or higher gauge needle, in particular a 36G-needle.

The frame 501 of the filling system 500A includes the IDDS placement fixture 600 for holding the IDDS 200. The IDDS placement fixture 600 is aligned with a length of the needle 455. The IDDS placement fixture 600 is further located at a distance from the needle placement fixture 605 sufficient to accommodate a first distance greater than a length of the needle 455 in a first position, and to accommodate a second distance less than the length of the needle 455 in a second position, so that the needle 455 is advanced into the IDDS 200. The location of each fixture 600, 605 relative to the first and second distance can be determined by, for example, the H-frame 501 as shown in the figures.

The drug filling system 500A further includes an outlet 560 configured to be open to an exterior of the drug filling system 500A, preferably an outlet located in the IDDS placement fixture 600 for overflow from the porous membrane 224 of the IDDS 200.

The needle placement fixture 605 and the IDDS placement fixture 600 are in moveable relation opposite to each other. For example, the needle placement fixture 605 can be fixed in place, and the IDDS placement fixture 600 can be moveable from the first to the second distance; or the needle placement fixture 605 can be moveable from the first to the second distance, and the IDDS placement fixture 600 can be fixed; or both the needle holder 605 and the IDDS placement fixture 600 can be moveable with respect to each other. The needle placement fixture 605 may be moveable from the first to the second distance in the frame 501 relative to the IDDS placement fixture 600.

The filling fixture 500A aligns the needle 455 over the septum 238 of the IDDS 200 but is at a fixed distance from the IDDS 200 itself, defined by the shoulder or rim 560A3. This dimension is fixed by the distance between the fixture shoulder or rim, and the tip of the needle 455. The IDDS 200 can be located such that when the needle 455 is filling the IDDS 200, the drug formulation, e.g., a solution, suspension, or gel as described herein can flow or be otherwise displaced through the porous surface 224 to the exterior of the filling system 500A, for example, through the drainage port 560, and be drained or wicked away to help ensure adequate wetting of the porous surface 224.

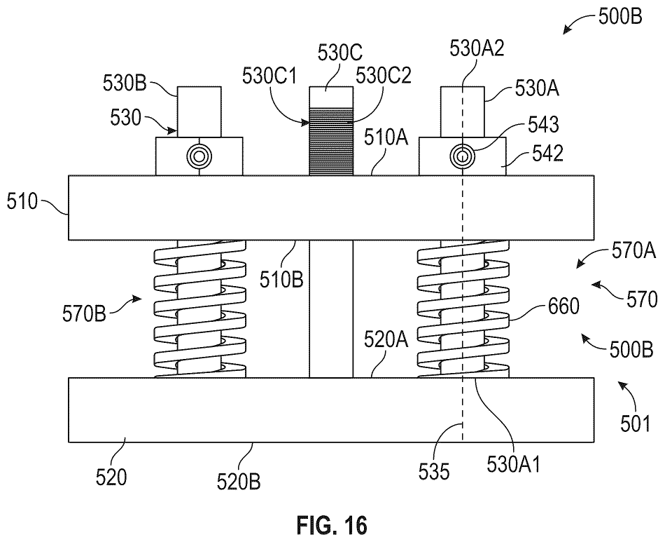

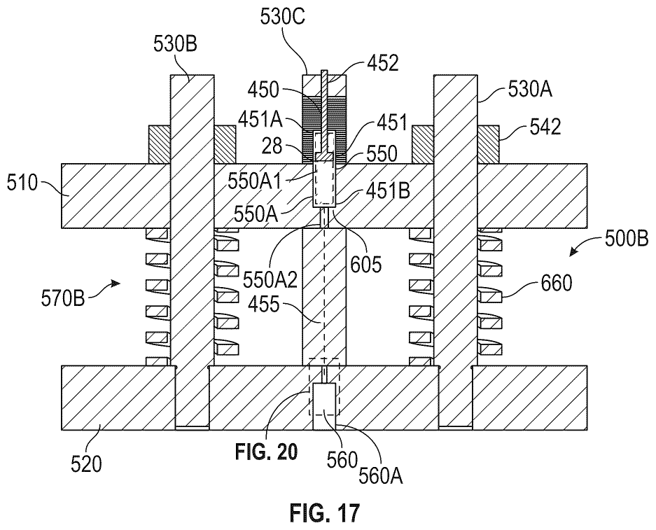

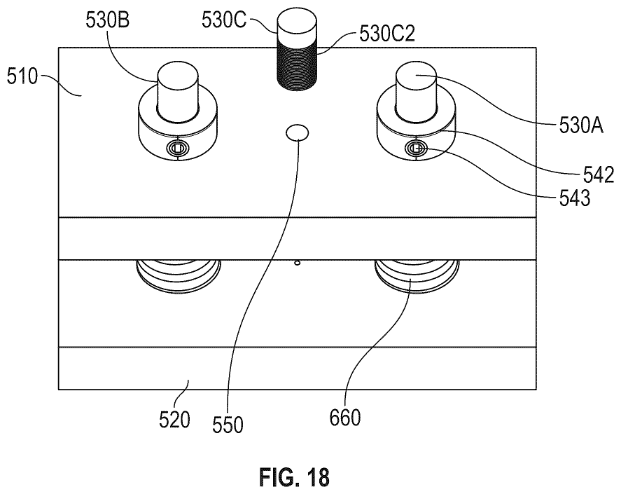

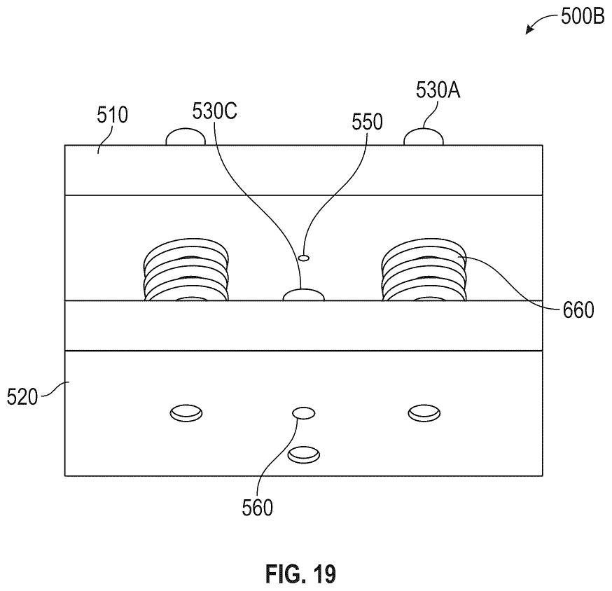

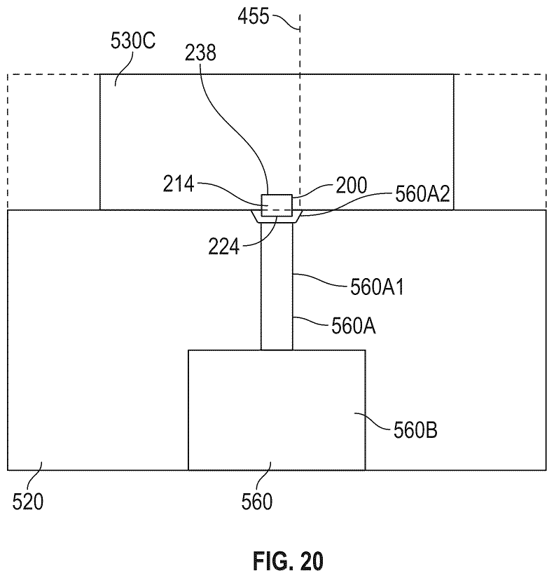

Turning to FIGS. 16-20, a second embodiment of a filling system (or drug filling system) 500B is shown. The filling system 500B is generally shown as an H-frame configuration 501 with top and bottom members 510, 520, and support posts 530, as indicated with the first embodiment 500A. The filling system 500B supports a syringe 450 (FIG. 17) with a drug 28 and an implantable drug delivery system (IDDS) 200 (FIG. 20). Though the IDDS 200 is identified in these and later figures, any IDDS disclosed herein may be utilized with the disclosed filling systems 500B.

The syringe 450 (FIG. 17) has a barrel 451 extending from a barrel top 451A to a barrel bottom 451B, a plunger 452 extending from the barrel top 451A and a needle 455 extending from the barrel bottom 451B. The IDDS 200 (FIG. 20) defines a compartment 214 with opposite ends 211, 213 (FIG. 7), with one end having a porous membrane 224 and another end having a septum 238 that is configured to receive a drug 28 from the needle 455 of the syringe 450.

As shown in FIGS. 16-19, the filling system 500B includes the top member 510 and the bottom member 520. The top and bottom members 510, 520 may be plates and more specifically rectangular or square plates, as nonlimiting examples, with respective top surfaces 510A, 520A and bottom surfaces 510B, 520B.

The top member 510 is configured to support the syringe 450. The bottom member 520 is configured to support the IDDS 200, such that the needle 455 of the syringe 450 faces the septum 238 of the IDDS 200. The top member 510 is configured to move toward and away from the bottom member 520, to insert the needle 455 into the IDDS 200 via the septum 238 of the IDDS 200. From this configuration, the compartment 214 of the IDDS 200 may be filled with the drug 28 upon activation of the plunger 452.

A first post 530A (or generally a post 530) extends from a bottom 530A1 to a top 530A2. The bottom 530A1 of the first post 530A is located at the bottom member 520, and may be fastened to the bottom member 520. The first post 530A extends through the top member 510 so the top 530A2 of the first post 530A is above the top member 510. The first post 530A defines a first axis 535. The top member 510 is configured to move toward and away from the bottom member 520 along the first axis 535, to insert the needle 455 into the IDDS 200 via the septum 238 of the IDDS 200.

More specifically, the system 500B includes plurality of posts 530, e.g. first and second posts 530A, 530B, extending from the bottom member 520, through the top member 510. The posts 530 may have a same shape and size as each other, as a non-limiting embodiment. The posts 530 may be oriented parallel to each other, and spaced apart from each other within the system 500B. From this configuration, the top member 510 is configured to move toward and away from the bottom member 520 along the first axis 535, as supported by the posts 530, to insert the needle 455 into the IDDS 200 via the septum 238 of the IDDS 200.

The top member 510 includes a top aperture (or first channel) 550 that extends through the top member 510, i.e., parallel to the first axis 535, defined by a top aperture wall 550A that may be cylindrically shaped. The top aperture 550 is configured to receive and fixedly seat the syringe 450 such that the needle 455 of the syringe 450 extends toward the second member 520.

The bottom member 520 has a bottom aperture 560 (or second channel), defined by a bottom aperture wall 560A that may be cylindrically shaped. The bottom aperture 560 extends parallel to the first axis 535. The bottom aperture 560 defines a drain passage or port for wet-out effluent.

A first stop system 570A (or generally a stop system 570) is secured to the first post 530A. The first stop system 570A limits motion of the top member 510 toward the bottom member 520. The first stop system 570A also supports the top member 510 relative to the bottom member 520. As shown, the system 500B includes a plurality of stop systems 570 including the first stop system 570A and a second stop system 570B that is secured to the second post 530B. The first and second stop systems 570A, 570B may be the same as each other so that the discussion herein shall focus on the first stop system 570A.

The plurality of posts 530 includes a marker post (or guide post) 530C. A top portion 530C1 of the marker post 530C includes position markers 530C2. The position markers 530C2 are utilized for identifying a position of the top member 510 relative to the bottom member 520. The position markers 530C2 may be utilized when the top member 510 is moved relative to the bottom member 520 to insert the needle 455 into the IDDS 200 via the septum 238 of the IDDS 200. The markers 530C2 may be utilized as a reference, e.g., relative to the top surface 510A of the top member 510.

The top aperture 550 of the top member 520 defines a top portion 550A1 that is cup-shaped and sized to receive and seat the barrel 451 of the syringe 450. A bottom portion 550A2 of the top aperture 550 is narrower, e.g., in diameter, than the top portion 550A1. The bottom portion 550A2 of the top aperture 550 is sized for the needle 455 of the syringe 450 to extend through the top member 510 when the barrel 451 of the syringe 450 is seated within the top portion 550A1 of the aperture 550.

The first stop system 570A includes a compression spring (or biasing member) 660 extending upwardly from the bottom member 520 toward the top member 510. A clamp (or lock fastener) 542 is secured via a fastener 543 (or set screw) to the first post 530A, above the top member 510, to react biasing motion from the compression spring 660. From this configuration, the first member 510 is vertically locked against the first post 530A. As shown in the figures for this embodiment, the compression spring 660 may be positioned to surround the first post 530A. As indicated, the second stop system 570B engages the second post 530B and is configured the same as the first stop system 570A.

Turning to FIG. 20, the bottom aperture 560 has a top portion 560A1 with a top edge 560A2 defining a top chamfer. A bottom portion 560B of the bottom aperture 560 is larger, e.g., in diameter, than the top portion 560A1 of the bottom aperture 560. With this configuration, the top edge 560A2 defines a seat for securely seating a portion of the IDDS 200 within the bottom aperture 560 of the bottom member 520, while providing a port for wet-out effluent.

With the above second embodiment, in use, the needle 455 can be located over the septum 238 of the IDDS 200, on the moveable frame 501, which can be supported by springs 660 and displaced via, e.g., use of a press positioned above the top plate 510. After pressing to a specific height offset from the IDDS 200, a set screw 543 applied to each lock 542 can lock the frame 501 into place. The configuration of the bottom plate 520 can align the IDDS 200 relative to the needle 455.

Use of the filling the IDDS 200 to allow the drug 28 to flow across the porous surface 224 can help ensure proper wetting of the porous surface 224. Machined features, i.e., spring-loaded posts 660 that control the positioning of the members 510, 520 relative to each other, in tandem with each lock 542 and set screw 543, can assist with repeatability, distending the needle 455 from where it is attached to the frame 501 to help ensure accurate filling of the IDDS 200 with a drug 28. The H-frame 501 of the drug filling system 500B can be configured and dimensioned for filling of an IDDS 200 of between about 1 to about 5 millimeters in diameter and between about 1 to about 50 millimeters long, with cavity/compartment volumes of between about 1 to about 1,000 microliters.

It is to be appreciated that various aspects of the above disclosed first and second embodiments may be interchanged with each other without falling outside the scope of the disclosure.

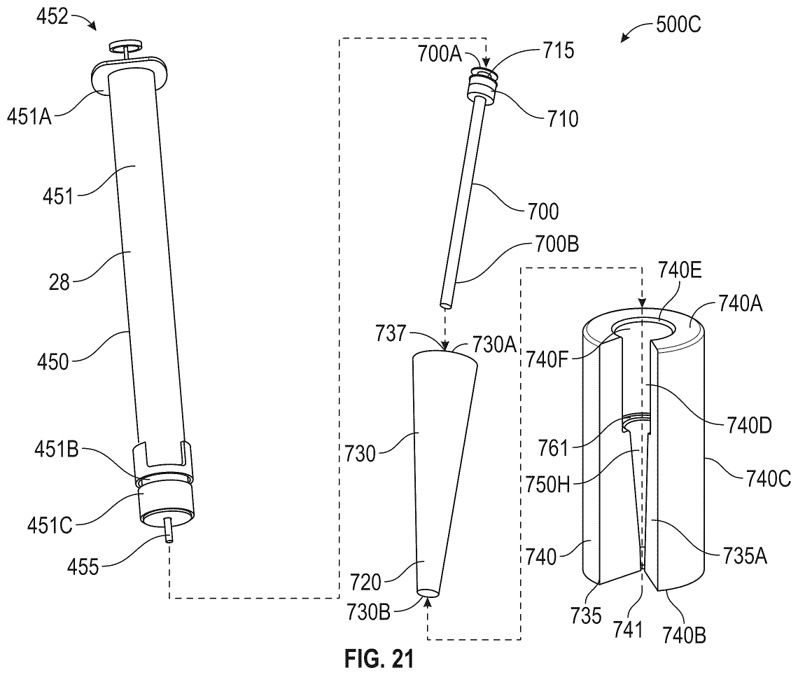

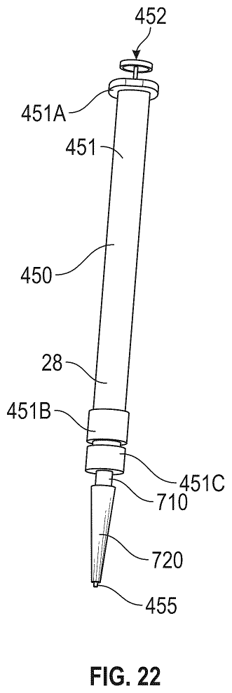

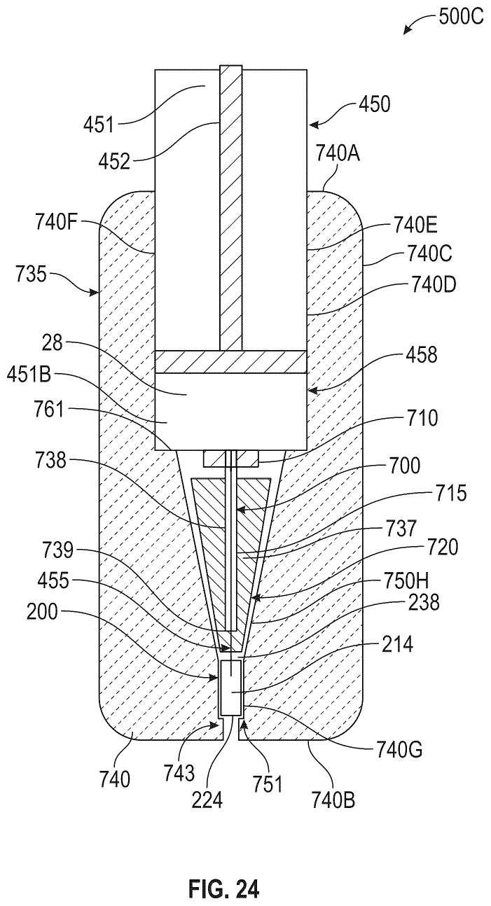

Turning to FIGS. 21-24, a third embodiment of the filling system 500C is illustrated. The filling system 500C supports a syringe 450 with a drug 28 and an implantable drug delivery system (IDDS) 200 (FIG. 24). The syringe 450 has a barrel 451 extending from a barrel top 451A to a barrel bottom 451B, a plunger 452 extending from the barrel top 451A and a needle 455 extending from the barrel bottom 451B. The barrel bottom 451B is shown as being capped by an end cap 451C, though this is not intended on limiting the scope of the embodiments. The IDDS 200 defines a compartment 214 with opposite ends 211, 213 (FIG. 7), with one end having a porous membrane 224 and another end having a septum 238 that is configured to receive the drug 28 from the needle 455 of the syringe 450.

The filling system 500C includes a filling fixture 735, defined by a fixture wall 740 that may have a generally cylindrical shape, extending from a top 740A to a bottom 740B along a fixture axis 741. An outer wall surface 740C and inner wall surface 740D are radially spaced from each other. The inner wall surface 740D of the filling fixture 735 defines an inner wall contour or fixture passage (or channel) 740E that includes a top bore (or top passage) portion 740F (otherwise referred to as a syringe receiving bore) that is configured to seat the barrel bottom 451B of the syringe 450.

The top bore portion 740F may define a top bore shoulder 761 (or syringe seat) that keeps the syringe 450 from moving axially downward within the fixture 735. The top bore shoulder 761 may be defined by a step change in diameter between a bottom of the top bore portion 740F and a top of the conical bore portion 740H (discussed below). A radial size of the top bore portion 740F may be such that the barrel 451 slides without friction, as a nonlimiting example.

As shown in FIG. 24, a bottom bore (or bottom passage) portion 740G (otherwise referred to as an IDDS receiving bore) is configured to seat the IDDS 200 such that the septum 238 of the IDDS 200 faces the syringe 450. That is, the bottom bore portion 740G may define a bottom bore shoulder 751 (or IDDS seat) that keeps the IDDS 200 from moving axially downward within the fixture 735. The bottom bore shoulder 751 may be defined by a step change in diameter between a bottom of the bottom bore portion 740G and a top of the drain passage 743 (discussed below).

With the above disclosed configuration, the top bore portion 740F and the bottom bore portion 740 of the fixture passage 740E are spaced apart from each other such that the needle 455 of the syringe 450 extends into the IDDS 200. Below the bottom bore portion 740G, to the bottom end 740B of the fixture 735, the fixture passage 740E may define a drain passage 743 or port for wet-out effluent, which may have a cylindrical shape.

As generally shown in FIGS. 21-24, a sheath 700 may be provided that extends from a top end 700A to a bottom end 700B and has a cylindrical shape to define a center sheath channel 715. The sheath 700 is configured to slide over the needle 455 of the syringe 450 so that the top end 700A of the sheath 700 is disposed against the barrel bottom 451B of the barrel 451. The top end 700A of the sheath 700 may define an expanded base 700C (or collar) which may be removably connected or bonded to the barrel bottom 451B. The sheath 700 may be utilized to prevent damage to the needle 455 when the syringe 450 is inserted into the fixture 735. The sheath 700 is shown as being shorter than the needle 455 so that the needle 455 is capable of entering the IDDS 200 when the sheath 700 is utilized in the fixture 735.

A centering cone (or centering wedge) 720 is provided for centering the needle 455, e.g., surrounded by the sheath 700, as it moves toward the IDDS 200 in the fixture 735. The centering cone 720 has an outer cone surface 730 defining an outer cone or frustoconical shape with wide end (or top end) 730A and a narrow end (or bottom end) 730B. The centering cone 720 has a center passage (otherwise referred to as a center channel or wedge channel) 737 defined by a center channel wall 738 that may be cylindrically shaped, and extends along the fixture axis 741. With this configuration, the centering cone 720 is configured for being positioned around the sheath 7000 and the needle 455 of the syringe 450. The centering cone 720 has a cone length, between top and bottom ends 730A, 730B, such that when the top end 730A is positioned against or near the barrel bottom 451B of the syringe 450, the needle 455 is configured to extend past the bottom end 730B of the centering cone 720.

The top end 730A of the centering cone 720 may be removably bonded to the base 700C of the sheath 700, so that the sheath 700 and centering cone 720 remain in place during use with the fixture 735. The sheath 700 is shown as being shorter than the centering cone 720, though this is not intended on limiting the scope of the embodiments providing the needle 455 is capable of entering the IDDS 200 when the sheath 700 and centering cone 720 are utilized in the fixture 735. As shown in FIG. 24, the center passage 737 may define a bottom passage shoulder 739 that that engages the sheath bottom 700B of the sheath 700 to limit movement of the centering cone 720 toward the barrel 451 beyond a predetermined distance, e.g., to prevent direct engagement between the centering cone 720 and the barrel 451.

The fixture passage 740E of the filling fixture 735 may define a conical bore (or passage) portion 740H (otherwise referred to as a guide portion or a wedge receiving bore) between the top bore portion 740F and the bottom bore portion 740G. The shape of the conical bore portion 740H is complementary to the outer cone shape of the centering cone 720. With this configuration, when the IDDS 200 is seated within the fixture 740, and the centering cone 720 is disposed against the syringe 455, the centering cone 720 is configured to guide the needle 455 into the IDDS 200.

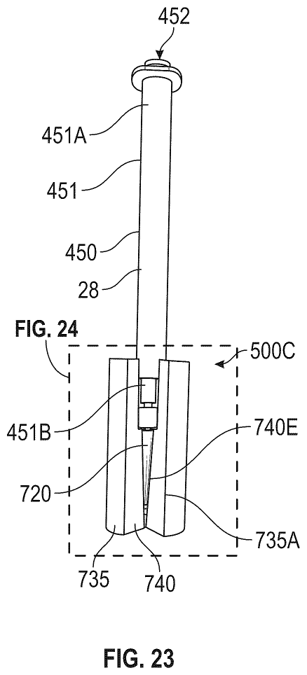

As shown in FIGS. 21 and 23, the wall 740 of the filling fixture 735 may define an axial slot 735A (or site-cutout) extending from the top end 740A to the bottom end 740B of the wall 740. That is, the slot 735A may define a circumferential gap in an otherwise full hoop shape of the filling fixture 735. The slot 735A provides visual access to the passage 740E for visually checking the process of filling loading the IDDS 200 and syringe 450 fitted with the sheath 700 and centering cone 720 into the fixture 735, and loading the drug 28 into the IDDS 200.

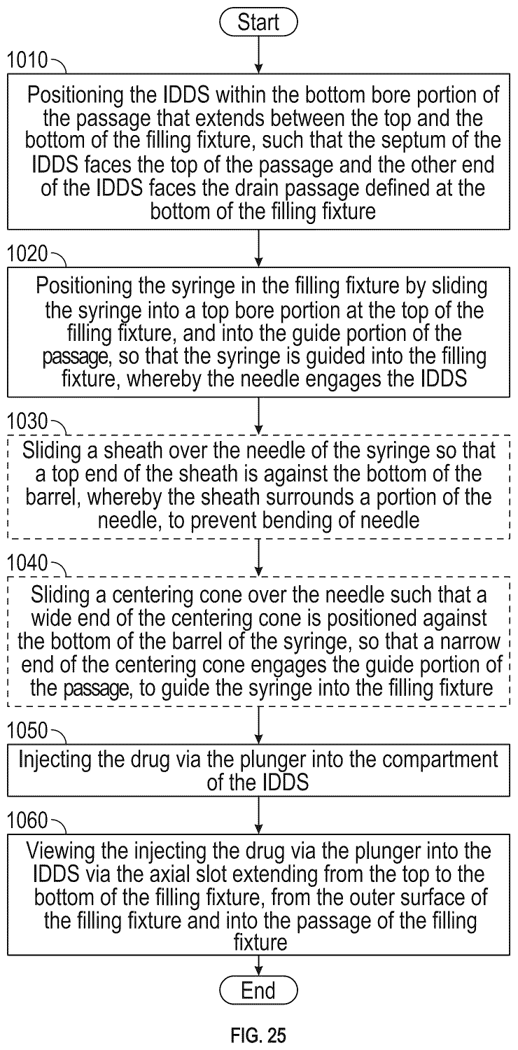

Turning to FIG. 25, a flowchart shows a method filling the IDDS 200 with the syringe 450 having the drug 28. In FIG. 25, boxes in dashed lines in the flowchart represent further explanations, including alternative embodiments, of one or more preceding steps and are not intended to limit the scope of the embodiments.

As shown in block 1010, the method includes positioning the IDDS 200 within the bottom bore portion 740G of the passage 740E that extends between the top 740A and the bottom 740B of the filling fixture 735. The IDDS 200 is positioned, such that the septum 238 of the IDDS 200 faces the top of the passage 740E and the porous membrane 214 of the IDDS 200 faces the drain passage defined at the bottom of the passage 740E.

As shown in block 1020, the method includes positioning the syringe 450 in the filling fixture 735 by sliding the syringe 450 into the top bore portion 740F at the top 740A of the filling fixture 735, and into the guide portion 761 of the passage 740E, so that the syringe 450 is guided into the filling fixture 735. From this action, the needle engages the IDDS 200.

As shown in block 1030 positioning the syringe 450 in the filling fixture 735 (block 1020) includes sliding a sheath 700 over the needle of the syringe 450 so that the top end 710 of the sheath 700 is against the bottom 451B of the barrel 451. From this action, the sheath 700 surrounds a portion of the needle 455, to prevent bending of needle 455.

As shown in block 1040, positioning the syringe 450 in the filling fixture 735 (block 1020) includes sliding the centering cone 720 over the needle 455 (i.e., the sheath 700 covered needle 455) such that the wide end 730A of the centering cone 720 is positioned against the bottom 451B of the barrel 451 of the syringe 450. With this configuration, the narrow end 730B of the centering cone 720 engages the guide portion 740H of the passage 740E, to guide the syringe 450 into the filling fixture 735.

As shown in block 1050, the method further includes injecting the drug 28 via the plunger 452 into the compartment 214 of the IDDS 200.

As shown in block 1060, the method further includes viewing the injecting the drug 28 via the plunger into the IDDS 200 via the axial slot 735A extending from the top 740A to the bottom 740A of the filling fixture 735, from the outer surface 740C of the filling fixture 735 and into the passage 740E of the filling fixture 735.

With the above embodiments, the IDDS placement fixture 735 can further include a component (e.g., the fixture 735) that acts as a needle guide within the passage 740E of the system 500C and above the septum 238 of the IDDS 200. The needle guide 735 can be in the form of the sheath 700 and/or the centering wedge 720, and have an opening 740E effective to guide the needle 455 into the septum 238. The needle guide 735 can provide additional support for the needle 455, for example, preventing bending during insertion into the septum 738. The opening 740E can have a conical configuration 740F at one end 740A, to provide additional guidance of the needle 455 into narrower portions 740G, 740H of the opening 740E of the needle guide 735. The needle guide 735 can further include a cut-out 735A for viewing the needle 455 and septum 238.

The outer configuration of the drug filling system 735 can be cylindrical, or have any other outer configuration, for example, pyramidal, a cuboid, or a rectangular prism. The fixture 735 of the drug filling system 500C may include a body having 740 a passage 740E having a first portion 740F configured for syringe placement. The fixture 735 can include a shoulder 761 or other feature, e.g., a ring-shaped rim, that provides a stop for the syringe barrel 451, and maintains the syringe 450 at a fixed distance from the IDDS 200. Placement of at least a portion of the body/barrel 451 of the syringe 450 within the fixture 735 allows greater stability of the syringe 735.

An additional component to support the needle 455 of the syringe 450 during insertion into the IDDS 200 can optionally be present. For example, as shown, the centering wedge 720, i.e., a needle guide including a sheath opening 738, can be placed in a second portion 740H of the passage 740E of the fixture 735 to provide buckling support and the guide sheath 700 for the needle 455. The opening 740E can be initially (at its top end) conical, and taper to a cylinder as described above. The fixture 735 and optional needle guide, e.g., centering wedge 720, holds and centers the needle 455 in relation to the septum 238 of the IDDS 200. The needle guide, e.g., a centering wedge 720. can be a separate component configured to fit the passage 740E of the fixture 735, or the fixture 735 can be manufactured to include the needle guide 720, e.g., the centering wedge 720, for example by machining or three-dimensional printing of the fixture 735. The drug filling system 500C is configured to control placement of a needle 455 used to fill the IDDS 200.

A section of the needle guide can include a cut-out 735A in the drug filling system 500C, which can be configured to allow the user to observe the needle 455 as it pierces the septum 238 of the IDDS 200. In one embodiment, the cut-out 735A is filled with a transparent material so that the outer wall 740 is a complete hoop.

The IDDS 200 for use with each drug filling system 500 disclosed herein has opposite ends 211, 213 (FIG. 7), which includes, e.g., at least one porous surface, e.g., a porous membrane 224 as described herein at the one end of the compartment 214 for a drug formulation 28, and a septum 238 at another end of the compartment 214. A porous surface, e.g., a porous membrane 224 as described herein can be located at an end of the compartment 214 opposite the septum 238. The drug filling systems 500 are configured to have a bottom opening, e.g., the drainage port, in fluid communication with the surface, e.g., the porous membrane 224 of the IDDS 200.

The disclosed systems solve the problem of alignment of the syringe with the IDDS so that drug-filling may be performed repeatedly, with precision and with minimal handling of and wear on the IDDS. All components of the different filling systems 500 may be made of rigid materials, such as metal. However this is not intended on limiting the scope of the embodiments.

It is to be appreciated that the porous surface 214 of the IDDS 200 has been identified herein with the discussion of various ones of the FIGS., however the outer shell 12A (e.g., FIG. 1), i.e., the sidewall of the IDDS 200, may be porous as indicated above. In one or more embodiments, the outer shell 12A is porous, and the ends 211, 213 are nonporous.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. “About” as used herein means the usual error range for measurement of the respective value readily known to the skilled person in this technical field, for example ±1%, or ±2%, or ±5% of the stated value. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. A filling system that supports a syringe with a drug and an implantable drug delivery system (IDDS),
wherein the syringe has a barrel extending from a barrel top to a barrel bottom, a plunger extending from the barrel top and a needle extending from the barrel bottom, and the IDDS defines a compartment with first and second ends that are opposite each other and a shell extending between the first and second ends, wherein:
one of the first and second ends has a septum that is configured to receive the drug from the needle of the syringe; and
one or more of another of the end first and second ends, and the shell, comprises a porous surface,
the filling system comprising:
a filling fixture having a wall extending from a top to a bottom along a first axis and defining an outer wall surface and inner wall surface that are radially spaced from each other,
wherein the inner wall surface defines a passage that includes:
a top bore portion that is configured to seat the barrel bottom of the syringe; and
a bottom bore portion configured to seat the IDDS such that the septum of the IDDS faces the syringe, wherein the top bore portion and the bottom bore portion are spaced apart from each other such that the needle of the syringe extends into the IDDS.

2. The filling system of claim 1, wherein:
the wall of the filling fixture defines an axial slot extending from the top to the bottom of the filling fixture to provide visual access to the passage.

3. The filling system of claim 1, further including:
a sheath extending from a top end to a bottom end and having a cylindrical shape,
wherein the sheath is configured to slide over the needle of the syringe so that the top end of the sheath is against the bottom of the barrel, whereby the sheath surrounds a portion of the needle, to prevent bending of needle, and wherein the needle is configured to extend past the bottom end of the sheath to engage the IDDS.

4. The filling system of claim 1, further including:
a centering cone having an outer cone surface defining an outer cone shape with wide end and a narrow end, and the centering cone defines a center passage such that the centering cone is configured for being positioned around the needle of the syringe;
wherein:
the centering cone has a cone length such that when the wide end is positioned against the bottom of the barrel of the syringe, the needle is configured to extend past the narrow end of the cone to engage the IDDS; and
the passage of the filling fixture has a conical bore portion with a conical shape between the top bore portion and the bottom bore portion that is complementary to the outer cone shape of the centering cone, such that when the IDDS is seated within the syringe and the centering cone is disposed against the syringe, the conical bore portion is configured to guide the centering cone into the filling fixture, and thereby guide the needle into the IDDS.

5. A method filling an implantable drug delivery system (IDDS) with a syringe having a drug,
wherein the syringe has a barrel extending from a barrel top to a barrel bottom, a plunger extending from the barrel top and a needle extending from the barrel bottom, and the IDDS defines a compartment with first and second ends that are opposite each other and a shell extending between the first and second ends, wherein:
one of the first and second ends has a septum that is configured to receive the drug from the needle of the syringe; and
one or more of another of the end first and second ends, and the shell, comprises a porous surface,
the method comprising:
positioning the IDDS within a bottom bore portion of a passage that extends between a top and a bottom of a filling fixture such that the septum of the IDDS faces the top of the passage and the other end of the ID DS faces a drain passage defined at the bottom of the filling fixture;
positioning the syringe in the filling fixture by sliding the syringe into a top bore portion at the top of the filling fixture, and into a guide portion of the passage, so that the syringe is guided into the filling fixture, whereby the needle engages the IDDS; and
the method further includes injecting the drug via the plunger into the compartment of the IDDS.

6. The method of claim 5, wherein positioning the syringe in the filling fixture includes:
sliding a sheath over the needle of the syringe so that a top end of the sheath is against the bottom of the barrel, whereby the sheath surrounds a portion of the needle, to prevent bending of needle, wherein the needle extends past the bottom end of the sheath.

7. The method of claim 5, wherein positioning the syringe in the filling fixture includes:
sliding a centering cone over the needle such that a wide end of the centering cone is positioned against the bottom of the barrel of the syringe, and a narrow end of the centering cone engages the guide portion of the passage and guides the syringe into the filling fixture, and wherein the needle extends past a narrow end of the cone.

8. The method of claim 5, comprising:
viewing the injecting the drug via the plunger into the IDDS via an axial slot extending from the top to the bottom of the filling fixture, from an outer surface of the filling fixture and into the passage of the filling fixture.

9. A filling system that supports a syringe with a drug and an implantable drug delivery system (IDDS),
wherein the syringe has a barrel extending from a barrel top to a barrel bottom, a plunger extending from the barrel top and a needle extending from the barrel bottom, and the IDDS defines a compartment with first and second ends that are opposite each other and a shell extending between the first and second ends, wherein:
one of the first and second ends has a septum that is configured to receive the drug from the needle of the syringe; and
one or more of another of the end first and second ends, and the shell, comprises a porous surface,
the filling system comprising:
a top member and a bottom member, wherein:
the top member is configured to support the syringe, and the bottom member is configured to support the IDDS, such that the needle of the syringe faces the septum of the IDDS; and
the top member is configured to move toward and away from the bottom member, to insert the needle into the IDDS via the septum of the ID DS, to thereby fill the compartment of the IDDS with the drug upon activation of the plunger.

10. The filling system of claim 9, including:
a first post extending from the bottom member, through the top member, wherein the first post defines a first axis, and the top member is configured to move toward and away from the bottom member along the first axis, along the first post, to insert the needle into the IDDS via the septum of the IDDS.

11. The filling system of claim 10, including:
a plurality of posts including the first post, extending from the bottom member, through the top member, wherein the posts are oriented parallel to each other, and spaced apart from each other within the system, and the top member is configured to move toward and away from the bottom member along the first axis, along the posts, to insert the needle into the IDDS via the septum of the IDDS.

12. The filling system of claim 10, wherein:
the top member includes a top aperture that extends through the top member along an axis parallel to the first axis;
the top aperture is configured to receive and seat the syringe such that the needle of the syringe extends toward the second member; and
the bottom member defines a bottom aperture, wherein the bottom aperture extends parallel to the first axis to define a drain passage.

13. The filling system of claim 12, including:
a first stop system, secured to the first post, that limits motion of the top member toward the bottom member and supports the top member relative to the bottom member.

14. The filling system of claim 13, wherein the first stop system includes:
a wedge, secured to the first post via a wedge bracket, having a wedge top and a wedge bottom, wherein:
the wedge top engages the top member of the filling system when the top member is moved toward the bottom member, to thereby limit motion of the top member toward the bottom member and support the top member relative to the bottom member.

15. The filling system of claim 12, comprising:
a syringe adapter configured for being removably inserted into the top aperture of the top member, wherein:
the syringe adapter defines a cup-shape with an adapter cavity that is sized to receive and seat the barrel of the syringe; and
the needle of the syringe extends through the bottom portion of the syringe adapter when the barrel of the syringe is seated within the syringe adapter.

16. The filling system of claim 12, comprising:
a bottom fixture defining a fixture center aperture that extends parallel to the first axis, wherein the fixture aperture is sized to receive the IDDS, and
wherein the bottom fixture is configured for being fixed against the bottom member so that the fixture center aperture is aligned with the bottom aperture.

17. The filling system of claim 11, wherein:
the plurality of posts includes a marker post, wherein a top portion of the marker post includes position markers for identifying a position of the top member relative to the bottom member when the top member is moved relative to the bottom member to insert the needle into the IDDS via the septum of the IDDS.

18. The filling system of claim 12, wherein the top aperture of the top member defines:
a top portion that is sized to receive and seat the barrel of the syringe; and
a bottom portion that is narrower than the top portion of the top aperture and sized for the needle of the syringe to extend through the top member when the barrel of the syringe is seated in the top portion of the top aperture.

19. The filling system of claim 12, wherein the bottom aperture of the bottom member defines:
a top portion, with a top edge defining a top chamfer; and
a bottom portion of that is larger than the top portion of the bottom aperture,
whereby the top edge defines a seat for seating a portion of the IDDS within the bottom aperture of the bottom member, thereby securely positioning the IDDS within the filling system.

20. The filling system of claim 13, wherein the first stop system includes:
a biasing member extending upwardly from the bottom member toward the top member; and
a clamp secured to the first post to react biasing motion from the biasing member, and thereby lock the first member against the first post.
